(19) United States
(12) Patent Application Publication
(10) Pub. No.: US 2014/0094380 A1
(43) Pub. Date: Apr. 3, 2014

(54) Methylation Biomarkers for Diagnosis of Prostate Cancer

(71) Applicants: Gavin J. Sherlock, Stanford, CA (US); James Duane Brooks, Stanford, CA (US); Richard M. Myers, Huntsville, AL (US); Yuya Kobayashi, Menlo Park, CA (US); Devin M. Absher, Huntsville, AL (US)

(72) Inventors: Gavin J. Sherlock, Stanford, CA (US); James Duane Brooks, Stanford, CA (US); Richard M. Myers, Huntsville, AL (US); Yuya Kobayashi, Menlo Park, CA (US); Devin M. Absher, Huntsville, AL (US)

(73) Assignee: THE BOARD OF TRUSTEES OF THE LELAND STANFORD JUNIOR UNIVERSITY, Palo Alto, CA (US)

(21) Appl. No.: 14/008,480

(22) PCT Filed: Apr. 2, 2012
(86) PCT No.: PCT/US2012/031876

§ 371 Date: Dec. 10, 2013

Related U.S. Application Data
(60) Provisional application No. 61/471,545, filed on Apr. 4, 2011.

(51) Int. Cl. C12Q 1/68

(52) U.S. Cl. CPC C12Q 1/6886
USPC 506/9; 435/6.11

(57) ABSTRACT

Biomarkers for diagnosis and prognosis of prostate cancer are provided. The biomarkers are promoter sequences have altered methylation patterns relative to normal prostate tissue. Altered expression of DNA methyltransferases (DNMT) and proteins that interact with DNMT result in increased methylation at a subset of prostate tumor hypermethylation sites.

### GOVERNMENT RIGHTS

This invention was made with Government support under contract CA111782 awarded by the National Institutes of Health. The Government has certain rights in this invention.

### BACKGROUND OF THE INVENTION

Identification of differentially altered genomic sequences also furthers the understanding of the progression and nature of complex diseases such as cancer, and is key to identifying the genetic factors that are responsible for the phenotypes associated with development of, for example, the metastatic phenotype. Identification of copy number alterations in various types of cancers can both provide for early diagnostic tests, and further serve as therapeutic targets.

Early disease diagnosis is of central importance to halting disease progression, and reducing morbidity. Analysis of a patient's tumor provides the basis for more specific, rational cancer therapy that may result in diminished adverse side effects relative to conventional therapies. Furthermore, confirmation that a tumor poses less risk to the patient (e.g., that the tumor is benign) can avoid unnecessary therapies.

Prostate cancer is the most commonly diagnosed malignancy for men in the United States with an estimated 217,730 new cases projected for 2010. After more than two decades of widespread serum prostate specific antigen (PSA) testing, clinical prostate cancer has shifted to a predominantly localized disease. However, two large-scale, randomized trials of PSA screening suggest that prostate cancer is over-diagnosed and over-treated, likely because many cancers that are detected are never destined to progress. However, prostate cancer can have an aggressive and lethal course and an estimated 32,050 men are projected to die of prostate cancer in 2010. This broad range of clinical behavior is likely a reflection of the underlying genomic diversity of the tumors. Previous studies of prostate tumors reported significant heterogeneity in the gene expression profiles and genomic structural alterations including DNA copy number changes and gene fusions often involving the ETS family of transcription factors detectable in approximately half of prostate tumors. However, exon sequencing of known oncogenes and tumor suppressors has found few somatic mutations and the calculated background mutation rate appears to be relatively low. This suggests the presence of other forms of genomic aberrations that contribute to the observed gene expression variations, and in turn, the diversity in tumor behavior.

Conventional screening for prostate cancer utilizes the prostate specific antigen (PSA) blood test, and the digital rectal exam (DRE). PSA is an enzyme produced in the prostate that is found in the seminal fluid and the bloodstream. An elevated PSA level in the bloodstream does not necessarily indicate prostate cancer, since PSA can also be raised by infection or other prostate conditions such as benign prostatic hyperplasia (BPH). Many men with an elevated PSA do not have prostate cancer. Nonetheless, a PSA level greater than 4.0 nanograms per milliliter of serum was established initially as the cutoff where the sensitivity for detecting prostate cancer was the highest and the specificity for detecting non-cancerous conditions was the lowest. A PSA level above 4.0 ng per milliliter of serum may trigger a prostate biopsy to search for cancer. The digital rectal exam is usually performed along with the PSA test, to check for physical abnormalities that can result from tumor growth.

The PSA test is an imperfect screening tool. A man can have prostate cancer and still have a PSA level in the “normal” range. Approximately 25% of men who are diagnosed with prostate cancer have a PSA level below 4.0. In addition, only 25% of men with a PSA level of 4-10 are found to have prostate cancer. With a PSA level exceeding 10, this rate jumps to approximately 65%.

Current methods of diagnosis and prognostication of prostate cancer are inadequate, because most prostate tumors present with low PSA, intermediate grade and early stage. Improved markers are of interest. The present invention addresses these needs.

### SUMMARY OF THE INVENTION

The present invention relates to the identification of novel biomarkers for diagnosis and prognosis of prostate cancer. The biomarkers of the invention are promoter sequences that have altered methylation patterns relative to normal prostate tissue, as set forth, for example, in Table 1, which lists genes shown herein to have hypermethylated or hypomethylated promoter regions. While the vast majority showed hypermethylation; 4 of the biomarkers set forth in Table 1 were hypomethylated: FCRL3, DARC, SCGB2A2, URB. In other embodiments of the invention, it is shown that altered expression of DNA methyltransferases (DNMT) and proteins that interact with DNMT result in increased methylation at a subset of prostate tumor hypermethylation sites.

In some embodiments of the invention, the methylation status of one or a plurality of biomarkers set forth in Table 1 is determined in a patient sample suspected of comprising prostate cancer cells; wherein increased methylation at the indicated biomarker is indicative of prostate cancer. In some embodiments, a plurality of markers is assessed, including 2, 5, 10, 15, 20, 25, 30, 35, 40, 45, 50, 55, 60 or more biomarkers are evaluated for hypermethylation. In some embodiments, the biomarker is other than GSTP1 or CDKN2.

In some embodiments the patient sample is a tumor biopsy. In other embodiments the patient sample is a convenient bodily fluid, for example a blood sample, urine sample, and the like. The biomarkers of the present invention may further be combined with other biomarkers for prostate cancer, including without limitation prostate specific antigen, chromosome copy number alterations, and the like.

In other embodiments, molecular assays are provided that determine the methylation status of one or more biomarkers set forth in Table 2 to identify a risk classification for a prostate cancer patient. For example, patients may be stratified using methylation status of one or more genes associated with cancer recurrence or death from cancer.

### BRIEF DESCRIPTION OF THE DRAWINGS

FIG. 1. Hierarchical clustering of prostate tissues by DNA methylation. Unsupervised hierarchical clustering of 181 prostate tissues and 26,333 CpGs, by sample and by CpG. Red branches represent tumor samples and blue branches represent benign adjacent samples. Red pixels represent high DNA methylation while green pixels represent low DNA methylation.

FIG. 2. Differentially methylated CpGs of prostate tumors. Unsupervised hierarchical clustering of 181 prostate tissues based on the 5,912 and 2,151 CpG sites hypermethylated and hypomethylated in prostate tumors, respectively, as identified by 2-class SAM. Red branches represent tumor samples and blue branches represent benign adjacent samples. Red pixels represent high DNA methylation while green pixels represent low DNA methylation.

FIG. 3. GSTP1 CpG island hypermethylation in prostate tumors. (A) Diagram of the RefSeq annotation of the GSTP1 gene. The green box represents a CpG island calculated by UCSC Genome Browser. Circles are CpG sites assayed by HumanMethylation27: red circles represent probes that were identified to be hypermethylated in prostate tumors by 2-class SAM, the green circle represents a probe that was hypomethylated, and the gray circle represents a probe that showed no significant change. The numbers below the circles indicate the relative distance in base pairs from the predicted TSS. (8) Heatmap depicts DNA methylation pattern of the 7 probes near GSTP1. The dendrogram is based on the hierarchical clustering from FIG. 2. Red branches represent tumor samples and blue branches represent benign adjacent samples. Coordinates are based on NCBI36/hg18 human genome assembly.

FIG. 4. Expression of DNMTs and EZH2 correlates with global hypermethylation in prostate tumors. Comparison of transcript levels of DNMTs and EZH2 measured by TaqMan qPCR with the average DNA methylation levels of CpG sites that are hypermethylated in prostate tumors. Blue circles are benign adjacent samples and red circles are tumor samples. P-value was calculated by linear regression analysis. Y-axis: average DNA methylation levels (beta score). X-axis: relative gene expression levels [log₂(RQ)]. Black line: linear regression. (A) DNMT1 expression. (8) DNMT3A expression. (C) DNMT3A2 expression. (D) DNMT38 expression. (E) EZH2 expression. (F) Comparison of DNMT and EZH2 transcript levels between benign adjacent tissues (blue) and tumors (red). Significant differences are indicated by asterisks; P values were calculated by t-test. Standard errors are depicted by error bars. Y-axis: relative gene expression levels [log₂(RQ)].

FIG. 5. Overexpression of DNMTs and EZH2 results in increased methylation at a subset of prostate tumor hypermethylation sites. Ideal (black) and empirical (red) cumulative distribution functions of change in DNA methylation after DNMT or EZH2 transfection into cultured normal prostate cells. The empirical distribution functions are based on the 5,912 CpGs that were hypermethylated in prostate tumors, while the ideal distribution functions are based on all 26,333 CpGs assayed on the array. Overexpression of (A) DNMT3A, (8) DNMT3A2, (C) DNMT381, (D) DNMT382, (E) DNMT383, (F) EZH2, (G) DNMT3A and EZH2, (H) DNMT3A2 and EZH2, (I) DNMT381 and EZH2, (J) DNMT382 and EZH2, and (K) DNMT383 and EZH2.

FIG. 6. Unpaired 2-class SAM comparing benign adjacent prostate tissues and tumors. Benign adjacent vs tumor unpaired 2-class SAM analysis of the 181 prostate samples. False discovery rate of 0.78% resulted in 8,063 differentially methylated CpGs including 5,912 hypermethylated CpGs (red) and 2,151 hypomethylated CpGs (green).

FIG. 7. Paired 2-class SAM comparing benign adjacent prostate tissues and tumors. Benign adjacent vs tumor paired 2-class SAM analysis of the 181 prostate samples. False discovery rate of 0.78% resulted in 7,741 differentially methylated CpGs including 5,556 hypermethylated CpGs (red) and 2,185 hypomethylated CpGs (green).

FIG. 8. APC proximal promoter hypermethylation in prostate tumors. (A) Diagram of the RefSeq annotation of the APC gene. There are no CpG islands, calculated by the UCSC Genome Browser, in this window. Circles are CpG sites assayed by HumanMethylation27: red circles represent probes that were identified to be hypermethylated in prostate tumors by 2-class SAM. The numbers above and below the circles indicate the relative distance in base pairs from the predicted TSS. (8) Heatmap depicts DNA methylation pattern of the 6 probes near APC. The dendrogram is based on the hierarchical clustering from FIG. 2. Red branches represent tumor samples and blue branches represent benign adjacent samples. Coordinates are based on NCB136/hg18 human genome assembly.

FIG. 9. RASSF1 proximal promoter hypermethylation in prostate tumors. (A) Diagram of the RefSeq annotation of the RASSF1 gene. Green boxes represent the CpG islands calculated by UCSC Genome Browser. Circles are CpG sites assayed by HumanMethylation27: red circles represent probes that were identified to be hypermethylated in prostate tumors by 2-class SAM and the gray circles represent probes that showed no significant change. The numbers above and below the circles indicate the relative distance in basepairs from the predicted TSS. (8) Heatmap depicts DNA methylation pattern of the 9 probes near RASSF1. The dendrogram is based on the hierarchical clustering from FIG. 2. Red branches represent tumor samples and blue branches represent benign adjacent samples. Coordinates are based on NCB136/hg18 human genome assembly.

FIG. 10. Diagnostic markers of prostate cancer identified by PAM. Unsupervised hierarchical clustering of 181 prostate samples based on the 87 diagnostic CpG sites identified by PAM. Red branches represent tumor samples and blue branches represent benign adjacent samples. Red pixels represent high DNA methylation while green pixels represent low DNA methylation.

FIG. 11. PyroMark validates HumanMethylation27 results. PyroMark sequencing results compared to HumanMethylation27 beta scores at 9 diagnostic CpGs identified by PAM. Blue circles are benign adjacent samples and red circles are tumor samples. Y-axis: fraction methylation calculated from PyroMark. X-axis: fraction methylation calculated from HumanMethylation27 (beta scores). Black line: linear regression. (A) CYBA (cg19790294). (8) GDAP1L1 (cg04448487). (C) HIF3A (cg02879662). (D) LGLS1 (cg19853760). (E) LOC387758 (cg04622802). (F) MCAM (cg21096399). (G) RPIP8 (cg13102585). (H) RA833A (cg24340926). (I) SCG82A2 (cg22862656).

FIG. 12. Comparison of neighboring CpGs by PyroMark. PyroMark sequencing results comparing neighboring CpGs of the 9 diagnostic CpGs identified by PAM. Each diamond represents a CpG methylation level for an individual sample. Lines connect CpGs from each sample. Blue lines are benign adjacent samples, red lines are tumor samples. Y-axis: fraction methylation calculated from PyroMark. X-axis: relative coordinates in basepairs. Box indicates CpG assayed by HumanMethylation27. (A) CYBA (cg19790294). (B) GDAP1L1 (cg04448487). (C) HIF3A (cg02879662). (D) LGLS1 (cg19853760). (E) LOC387758 (cg04622802). (F) MCAM (cg21096399). (G) RPIP8 (cg13102585). (H) RA833A (cg24340926). (I) SCG82A2 (cg22862656).

### DETAILED DESCRIPTION OF THE EMBODIMENTS

Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### DEFINITIONS

As used herein, the term “methylation status” as applied to a gene refers to whether one or more cytosine residues present in a CpG context have or do not have a methylation group. Methylation status may also refer to the fraction of cells in a sample that do or do not have a methylation group on such cytosines. These cytosines are typically in the promoter region of the gene, though may also be found in the body of the gene, including introns and exons.

As used herein, the term “prostate cancer” is used interchangeably and in the broadest sense refers to all stages and all forms of cancer arising from the tissue of the prostate gland.

According to the tumor, node, metastasis (TNM) staging system of the American Joint Committee on Cancer (AJCC), AJCC Cancer Staging Manual (7th Ed., 2010), the various stages of prostate cancer are defined as follows: Tumor: TI: clinically inapparent tumor not palpable or visible by imaging, T1a: tumor incidental histological finding in 5% or less of tissue resected, T1b: tumor incidental histological finding in more than 5% of tissue resected, T1c: tumor identified by needle biopsy; T2: tumor confined within prostate, T2a: tumor involves one half of one lobe or less, T2b: tumor involves more than half of one lobe, but not both lobes, T2c: tumor involves both lobes; T3: tumor extends through the prostatic capsule, T1a: extracapsular extension (unilateral or bilateral), T3b: tumor invades seminal vesicle(s); T4: tumor is fixed or invades adjacent structures other than seminal vesicles (bladder neck, external sphincter, rectum, levator muscles, or pelvic wall). Node: NO: no regional lymph node metastasis; NI: metastasis in regional lymph nodes. Metastasis: M0: no distant metastasis; MI: distant metastasis present.

The Gleason Grading system is used to help evaluate the prognosis of men with prostate cancer. Together with other parameters, it is incorporated into a strategy of prostate cancer staging, which predicts prognosis and helps guide therapy. A Gleason “score” or “grade” is given to prostate cancer based upon its microscopic appearance. Tumors with a low Gleason score typically grow slowly enough that they may not pose a significant threat to the patients in their lifetimes. These patients are monitored (“watchful waiting” or “active surveillance”) over time. Cancers with a higher Gleason score are more aggressive and have a worse prognosis, and these patients are generally treated with surgery (e.g., radical prostectomy) and, in some cases, therapy (e.g., radiation, hormone, ultrasound, chemotherapy).

As used herein, the term “tumor tissue” refers to a biological sample containing one or more cancer cells, or a fraction of one or more cancer cells. Those skilled in the art will recognize that such biological sample may additionally comprise other biological components, such as histologically appearing normal cells (e.g., adjacent the tumor), depending upon the method used to obtain the tumor tissue, such as surgical resection, biopsy, or bodily fluids.

As used herein, the term “adjacent tissue (AT)” refers to histologically “normal” cells that are adjacent a tumor. For example, the AT expression profile may be associated with disease recurrence and survival.

Prognostic factors are those variables related to the natural history of cancer, which influence the recurrence rates and outcome of patients once they have developed cancer. Clinical parameters that have been associated with a worse prognosis include, for example, increased tumor stage, PSA level at presentation, and Gleason grade or pattern. Prognostic factors are frequently used to categorize patients into subgroups with different baseline relapse risks.

The term “prognosis” is used herein to refer to the likelihood that a cancer patient will have a cancer-attributable death or progression, including recurrence, metastatic spread, and drug resistance, of a neoplastic disease, such as prostate cancer. For example, a “good prognosis” would include long term survival without recurrence and a “bad prognosis” would include cancer recurrence.

The term “recurrence” is used herein to refer to local or distant recurrence (i.e., metastasis) of cancer. For example, prostate cancer can recur locally in the tissue next to the prostate or in the seminal vesicles. The cancer may also affect the surrounding lymph nodes in the pelvis or lymph nodes outside this area. Prostate cancer can also spread to tissues next to the prostate, such as pelvic muscles, bones, or other organs. Recurrence can be determined by clinical recurrence detected by, for example, imaging study or biopsy, or biochemical recurrence detected by, for example, sustained follow-up prostate-specific antigen (PSA) levels ≧0.4 ng/mL or the initiation of salvage therapy as a result of a rising PSA level.

The term “Prostate Cancer-Specific Survival (PCSS)” is used herein to describe the time (in years) from surgery to death from prostate cancer. Losses due to incomplete follow-up or deaths from other causes are considered censoring events. Clinical recurrence and biochemical recurrence are ignored for the purposes of calculating PCSS.

The term “nucleic acid” as used herein refers to a deoxyribonucleotide or ribonucleotide in either single- or double-stranded form. The term encompasses nucleic acids, i.e., oligonucleotides, containing known analogues of natural nucleotides that have similar or improved binding properties, for the purposes desired, as the reference nucleic acid. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs). PNAs contain non-ionic backbones, such as N-(2-aminoethyl)glycine units. Other synthetic backbones encompasses by the term include methylphosphonate linkages or alternating methylphosphonate and phosphodiester linkages and benzylphosphonate linkages. The term nucleic acid is used interchangeably with gene, DNA, polynucleotide, cDNA, mRNA, oligonucleotide primer, probe and amplification product.

The term a “nucleic acid array” as used herein is a plurality of target elements, each target element comprising one or more nucleic acid molecules (probes) immobilized on a solid surface to which sample nucleic acids are hybridized. The nucleic acids of a target element can contain sequence from specific genes or clones, such as the probes of the invention. Other target elements will contain, for instance, reference sequences. Target elements of various dimensions can be used in the arrays of the invention. Generally, smaller, target elements are preferred. Typically, a target element will be less than about 1 cm in diameter. Generally element sizes are from 1 μm to about 3 mm, preferably between about 5 μm and about 1 mm.

The target elements of the arrays may be arranged on the solid surface at different densities. The target element densities will depend upon a number of factors, such as the nature of the label, the solid support, and the like. One of skill will recognize that each target element may comprise a mixture of probe nucleic acids of different lengths and sequences. Thus, for example, a target element may contain more than one copy of a cloned piece of DNA, and each copy may be broken into fragments of different lengths. The length and complexity of the probe nucleic acid fixed onto the target element is not critical to the invention. One of skill can adjust these factors to provide optimum hybridization and signal production for a given hybridization procedure, and to provide the required resolution among different genes or genomic locations. In various embodiments, probe sequences will have a complexity between about 1 kb and about 1 Mb, between about 10 kb to about 500 kb, between about 200 to about 500 kb, and from about 50 kb to about 150 kb.

The term “sample of human nucleic acid” as used herein refers to a sample comprising human DNA in a form suitable for determination of methylation status of selected biomarkers. The nucleic acid may be isolated, cloned or amplified; and is typically genomic DNA or a product thereof, e.g. an amplified product of a chromosomal region following bisulfite conversion, etc. The nucleic acid sample may be extracted from particular cells or tissues. The cell or tissue sample from which the nucleic acid sample is prepared is typically taken from a patient suspected of having prostate cancer, usually a sample comprising the suspected neoplastic cells.

Methods of isolating cell and tissue samples are well known to those of skill in the art and include, but are not limited to tissue sections, needle biopsies, and the like. Frequently the sample will be a clinical sample derived from a patient, including sections of tissues such as frozen sections or paraffin sections taken for histological purposes. The sample can also be derived from supernatants or the cells themselves from cell cultures, cells from tissue culture and other media in which it may be desirable to detect chromosomal abnormalities or determine copy number. In some cases, the nucleic acids may be amplified using standard techniques such as PCR, prior to the hybridization. The sample may be isolated nucleic acids immobilized on a solid. The sample may also be prepared such that individual nucleic acids remain substantially intact.

Amplification refers to the process by which DNA templates are increased in number through multiple rounds of replication. Conveniently, polymerase chain reaction (PCR) is the method of amplification, but such is not required, and other methods, such as loop-mediated isothermal amplification (LIA); ligation detection reaction (LDR); ligase chain reaction (LCR); nucleic acid sequence based amplification (NASBA); multiple displacement amplification (MDA); C-probes in combination with rolling circle amplification; and the like may find use. See, for example, Kozlowski et al. (2008) Electrophoresis. 29(23):4627-36; Monis et al. (2006) Infect Genet Evol. 6(1):2-12; Zhang et al. (2006) Clin Chim Acta. 363(1-2):61-70; Cao (2004) Trends Biotechnol. 22(1):38-44; Schweitzer and Kingsmore (2001) Curr Opin Biotechnol. 12(1):21-7; Lisby (1999) Mol. Biotechnol. 12(1):75-99. As known in the art, amplification reactions can be performed in a number of configurations, e.g. liquid phase, solid phase, emulsion, gel format, etc.

Sequencing platforms include, but are not limited to those commercialized by: 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; Helicos BioSciences Corporation (Cambridge, Mass.) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058; Applied Biosystems (e.g. SOLiD sequencing); Dover Systems (e.g., Polonator G.007 sequencing); Illumina as described U.S. Pat. Nos. 5,750,341; 6,306,597; and 5,969,119; and Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference. Such methods and apparatuses are provided here by way of example and are not intended to be limiting.

### Diagnostic Methods

In general, prostate cancer is detected in a patient based on the presence of one or more differentially methylated biomarkers from the group set forth in Table 1 in a biological sample (such as blood, sera, seminal fluid, urine and/or tumor biopsies) obtained from the patient. In other words, the methylation status of the selected biomarkers indicates the presence or absence of prostate cancer cells in a patient sample.

Various methods for determining the methylation status of the biomarkers set forth herein may be employed for the purposes of the present invention. Profiling methods known in the art include, without limitation, techniques based on one or more of bisulfite conversion, digestion with methylation-sensitive restriction enzymes, and affinity purification of methylated DNA. As discussed in the Examples, hypermethylation of one or more CpG islands in promoter sequences of the genes set forth in Table 1 is indicative that a cell is a prostate cancer cell.

In some embodiments, bisulfite conversion is used to determine the methylation status of a biomarker. Methylated cytosine has roughly the same base-pairing characteristics as unmethylated cytosine, and is thus indistinguishable by standard sequencing approaches. To overcome this, genomic DNA is treated with sodium bisulfite under conditions that cause deamination of unmethylated cytosine to uracil, while leaving methylated cytosine intact. The converted DNA may be sequenced directly or amplified, where the uracil is then replaced with thymine. Analysis of the DNA product may be performed by any convenient sequencing method to quantify the extent of methylation at each cytosine.

In one embodiment of the invention a bead array-based analysis of DNA methylation is used to determine biomarker methylation status. Bisulfite-converted sample DNA is assayed with two primers, each labeled with a different fluorescent dye. One primer is designed to hybridize if the cytosine is methylated (and unconverted), whereas the other will only hybridize to a converted sequence. The two primers are used in a PCR reaction with a locus-specific methylation-insensitive primer. The ratio of the PCR products is ascertained using a bead array platform. This technique provides quantitative evaluation of specific cytosines and can process many samples in parallel.

Short or long oligonucleotide arrays also find use. In such assays a DNA sample if bisulfite converted, and hybridized to a an array of oligonucleotides that distinguish between a converted an unconverted sequence. To compare samples, each sample is hybridized to an array and the resulting signals are compared. For methylation analysis, a tiling design is useful, with equidistantly spaced probes across portions of a genome or an entire genome. Commercially available tiling arrays are available for the human genome, as well as for human promoters. Single-nucleotide polymorphism (SNP) arrays have probes that selectively bind to specific polymorphic sequences, thus providing genotype information based on relative hybridization to the polymorphic probes. Using SNP arrays for DNA methylation analysis allows the genotyping of methylated DNA that has been isolated from polymorphic individuals.

Alternatively, methylation-sensitive restriction endonucleases are useful in DNA methylation analysis. Many such enzymes are known in the art, and are often inhibited by methylation of their recognition site, although some specifically digest methylated DNA. Many variations of restriction enzyme-based methods may be used in conjunction with genomic analysis. Generally, comparisons are made between a sample treated with an enzyme or a cocktail of enzymes and an untreated control; between a sample treated with a methylation-sensitive enzyme compared with a control treated with a methylation-insensitive isoschizomer; or between two test samples, such as two tissue types or mutant and wild-type samples, both treated with the same enzyme.

Affinity purification may be performed to enrich for DNA methylated sequences, e.g. utilizing methyl-binding domain (MBD), which binds methylated CG sites. Alternatively, a commercially available monoclonal antibody that specifically recognizes methylated cytosine can be used to immunoprecipitate methylated DNA.

Some methods may entail determining a baseline value of methylation status in a normal control, or in a patient before administering a dosage of agent, and comparing this with a value for the test response, i.e. after treatment, in a patient sample, etc. A significant increase may include a value greater than the typical margin of experimental error in repeat measurements of the same sample, expressed as one standard deviation from the mean of such measurements of the methylation status of one or more biomarkers set forth herein in the sample. Measured values of in a patient may be compared with the control value.

In other methods, a control value (e.g., a mean and standard deviation) is determined from a control population of individuals who have known prostate cancer status, e.g. previously diagnosed; following treatment with a therapeutic agent; and the like. Measured values are compared with the control value.

In other methods, a patient who is not presently receiving treatment but has undergone a previous course of treatment is monitored for methylation status of a biomarker to determine whether a resumption of treatment is required, i.e. whether residual cancer cells are present.

One skilled in the art will recognize that there are many statistical methods that may be used to determine whether there is a significant relationship between a diagnostic or prognostic parameter of interest and methylation status of a biomarker as described herein. In certain embodiments, the correlation of methylation status of multiple biomarkers may be assessed. For this purpose, the correlation structures may be examined through hierarchical cluster methods.

Assays can provide for normalization by incorporating the methylation status of certain normalizing genes, which do not significantly differ in methylation status under the relevant conditions. Normalization can be based on the mean or median signal of all of the assayed biomarkers or a large subset thereof (global normalization approach). In general, the normalizing genes, also referred to as reference genes should be genes that are known not to exhibit significantly different methylation in prostate cancer as compared to non-cancerous prostate tissue, and are not significantly affected by various sample and process conditions, thus provide for normalizing away extraneous effects.

The methylation status data used in the methods disclosed herein can be standardized. Standardization refers to a process to effectively put all the biomarkers on a comparable scale. This is performed because some biomarkers will exhibit more variation than others. Standardization is performed by dividing each methylation value by its standard deviation across all samples for that biomarker. Hazard ratios are then interpreted as the relative risk of recurrence per 1 standard deviation increase in methylation.

### Kits

This invention also provides diagnostic kits for the detection of methylation status alterations in the target biomarkers. In a preferred embodiment, the kits include one or more hybridization probes, e.g. a bead based array, etc. to the target regions of the invention, and/or probes for amplification. The kits can additionally include bisulfite reagents and instructional materials describing when and how to use the kit contents. The kits can also include one or more of the following: various labels or labeling agents to facilitate the detection of the probes, reagents for the hybridization including buffers, sampling devices including fine needles, swabs, aspirators and the like, positive and negative controls and so forth.

### EXPERIMENTAL

DNA methylation profiles have not been compared on a large scale between prostate tumor and normal prostate, and the mechanisms behind these alterations are unknown. In this study, we quantitatively profiled 95 primary prostate tumors and 86 benign adjacent prostate tissue samples for their DNA methylation levels at 26,333 CpGs representing 14,104 gene promoters by using the Illumina HumanMethylation platform. A 2-class Significance Analysis of this dataset revealed 5,912 CpG sites with increased DNA methylation and 2,151 CpG sites with decreased DNA methylation in tumors (FDR<0.8%). Prediction Analysis of this dataset identified 87 CpGs that are the most predictive diagnostic methylation biomarkers of prostate cancer. By integrating available clinical follow-up data, we also identified 69 prognostic DNA methylation alterations that correlate with biochemical recurrence of the tumor. To identify the mechanisms responsible for these genome-wide DNA methylation alterations, we measured the gene expression levels of several DNA methyltransferases (DNMTs) and their interacting proteins by TaqMan qPCR and observed increased expression of DNMT3A2, DNMT3B, and EZH2 in tumors. Subsequent transient transfection assays in cultured primary prostate cells revealed that DNMT3B1 and DNMT3B2 overexpression resulted in increased methylation of a substantial subset of CpG sites that also showed tumor-specific increased methylation.

### Results

To explore the prostate DNA methylome, we profiled 95 primary prostate tumors and 86 benign adjacent prostate tissues, including 70 matched pairs, using the Illumina HumanMethylation27 microarrays. These tissue samples were harvested from men who underwent radical retropubic prostatectomy for clinically localized prostate cancer. Surgeries were performed between 1998 and 2007 and detailed clinical data, including follow-up and recurrence status were available in 96 patients (88%). Mean patient age, pre-operative serum PSA levels, clinical stage and pathological Gleason grade were compatible with the risk profiles of contemporary patients undergoing surgery for prostate cancer.

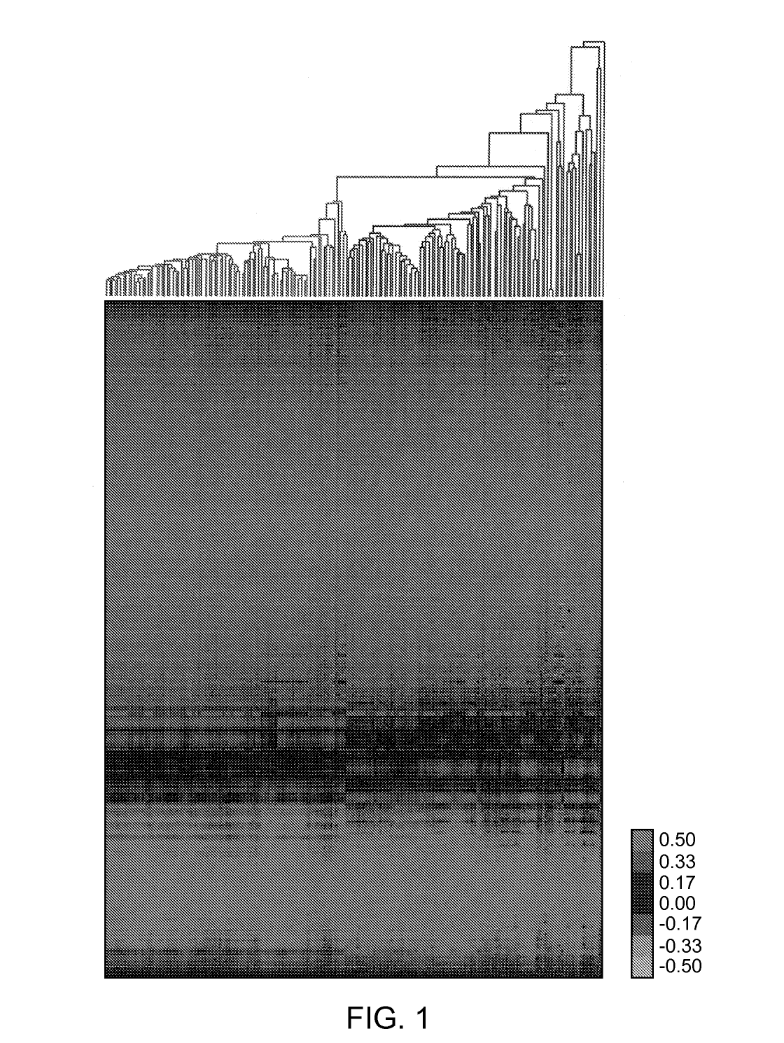

The Illumina HumanMethylation27 platform assays 27,578 CpG sites, almost all in the proximal promoter regions of 14,495 transcription start sites. After batch correcting and quality filtering the data, we were able to determine quantitative methylation status (beta scores; range: 0 to 1) for 26,333 CpG sites in 14,104 promoters. To investigate the similarities and differences of the DNA methylation profiles of the benign adjacent samples and tumor samples, as well as their heterogeneity, we performed unsupervised hierarchical clustering on the entire dataset (FIG. 1). When the data were clustered by sample, we observed two main clusters—one comprised almost entirely of benign adjacent samples (77/88) and the other comprised almost entirely of the tumor samples (67/71). The branch lengths in the benign adjacent sample cluster were generally shorter than the branch lengths in the tumor sample cluster, indicating more heterogeneity in methylation profiles among the tumor samples. Twenty-two of the samples did not fall into either of the two main clusters and formed long off-shooting branches or small clusters. Eighteen of these were tumor samples, further indicative of the heterogeneous nature of the tumor DNA methylome. By visual inspection, the majority of the samples showed relatively little methylation change between the tumor and benign adjacent clusters (FIG. 1), and most of these invariable CpG sites showed low levels of methylation in both benign adjacent and tumor samples. However, there were distinct CpG clusters with methylation patterns that distinguished the benign adjacent or tumor sample clusters, and, strikingly, a large number of CpG sites showed increased methylation in the tumor cluster compared to the benign adjacent cluster.

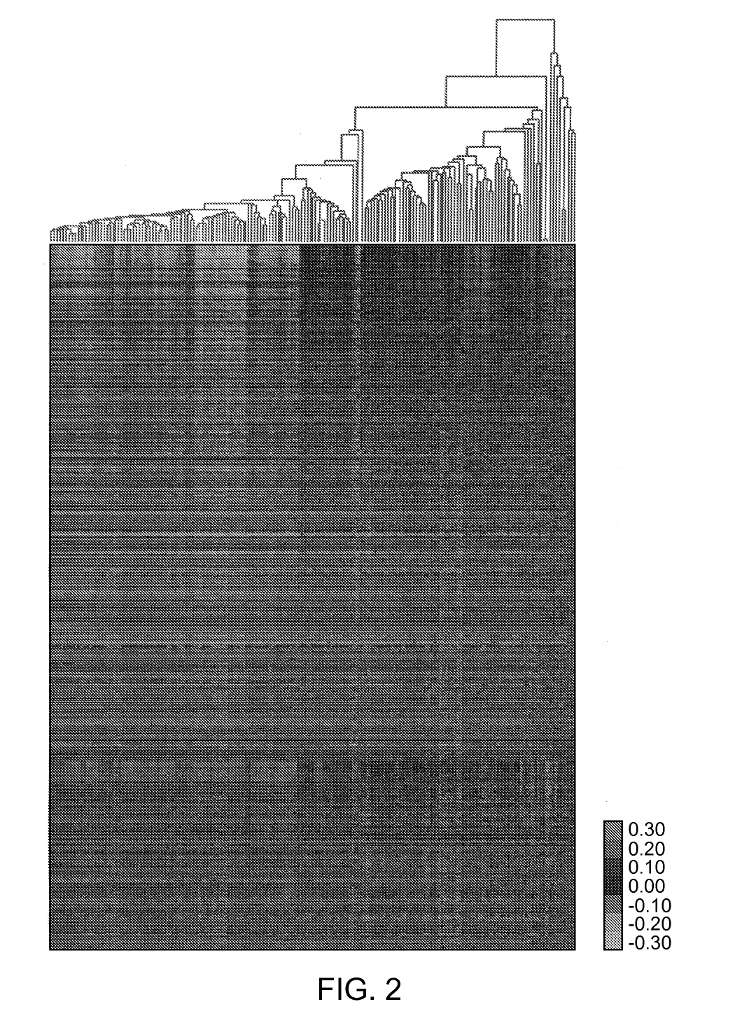

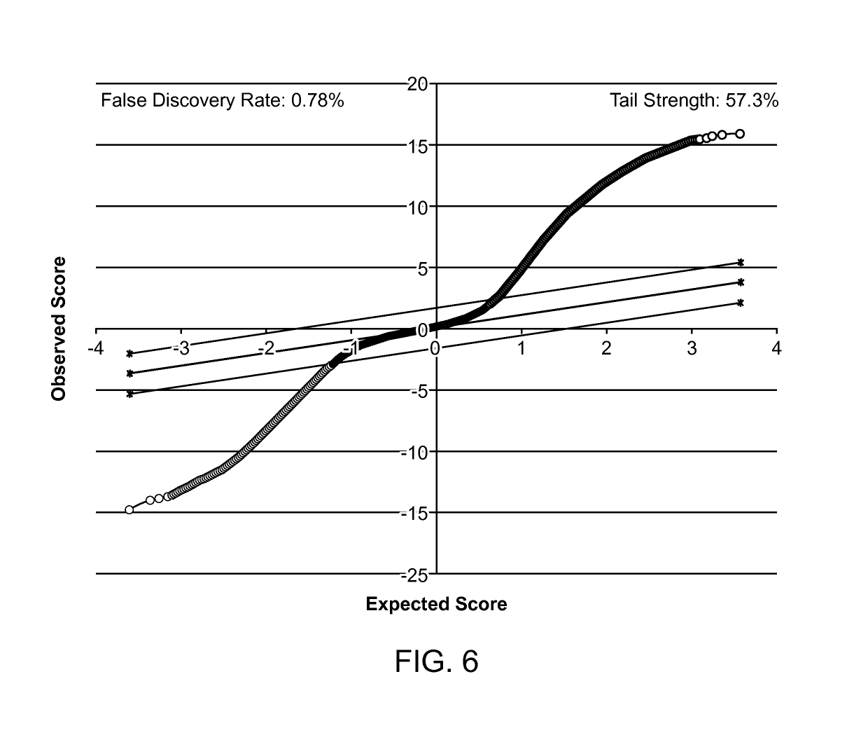

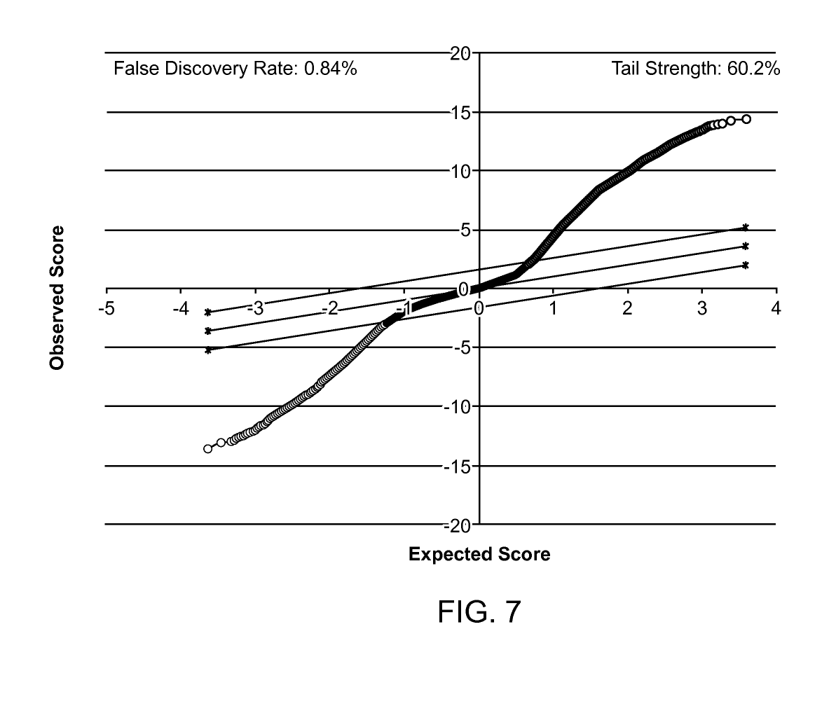

To identify the CpG sites with statistically different DNA methylation status between benign adjacent prostate tissues and tumors, we performed a two-class Significance Analysis of Microarrays (SAM). As we had matched benign adjacent tissues for only 70 of the 95 tumors used in this study, we conducted the SAM analysis as unpaired. The analysis identified 5,912 CpG sites hypermethylated in tumors compared to benign adjacent tissues, and 2,151 CpG sites hypomethylated at FDR<0.8% (FIG. 6). We performed hierarchical clustering on all samples based on these 8,063 differentially methylated CpG sites (FIG. 2). When the fold-change was examined for these sites, 1,851 sites had a 2-fold or greater change and as high as a 141-fold increase in methylation for a CpG near the transcriptional start site of ZNF342 (average normal beta: 5.30E-4, average tumor beta: 0.0756). All but 609 of the CpGs had a change of 5% or greater. While these 609 sites had a low level of fold-change, these were nonetheless identified as statistically significant changes that were detectable because of the large sample size (FIG. 6).

The 8,063 differentially methylated sites corresponded to 4,224 hypermethylated and 1,792 hypomethylated promoters. Of the 11,116 gene promoters represented by two or more CpG sites on the HumanMethylation27 platform, only 223 had opposite methylation effects (i.e., at least one hypermethylated CpG and at least one hypomethylated CpG). When the distances from transcriptional start sites were compared in these 223 promoters with opposite methylation effects, we saw enrichment for hypermethylated CpGs in the −100 bp to +800 bp range, whereas we saw enrichment for the hypomethylated CpGs in the −700 bp to −200 bp range. Thus, overall, nearly one third (8,063/26,333) of assayed promoter CpGs had a statistically significant change in DNA methylation, with most of those showing an increase in methylation. Interestingly, 43% (6,015/14,104) of all gene promoters assayed had at least one CpG with a tumor-specific methylation change. We repeated this analysis using two-class paired SAM on only the 70 matched sample pairs and observed similar results.

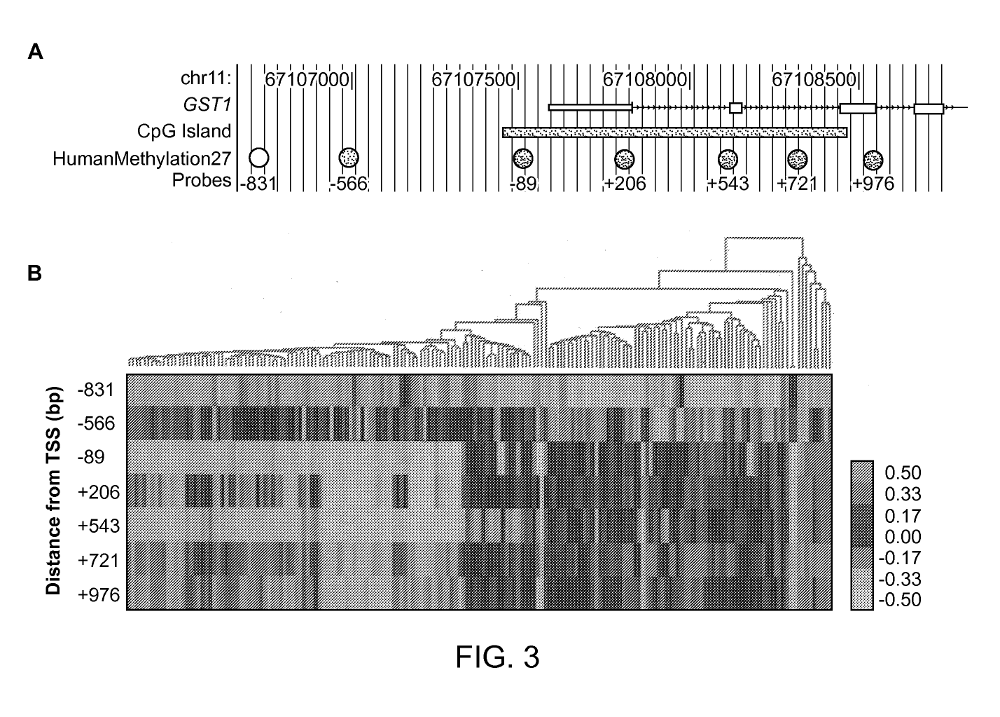

Diagnostic methylation markers. Among the CpG sites that we found to be differentially methylated in tumor versus benign adjacent prostate tissues by SAM, and shown clustered in FIG. 2, were several sites that had been previously characterized in prostate tumors, most notably several CpG sites near or within the GSTP1 gene. Hypermethylation of the CpG island overlapping the transcriptional start site of the GSTP1 gene has been associated with transcriptional silencing and is described as the most common molecular alteration in prostate cancer identified to date. Since GSTP1 promoter methylation is very common and specific for prostate cancer, many investigators have proposed using this methylation event as a diagnostic biomarker for prostate cancer. The HumanMethylation27 arrays contain seven CpG sites in the GSTP1 promoter. Five of these sites showed significantly increased DNA methylation in tumors, four of which are located in the promoter CpG island that had been previously characterized as a site of hypermethylation in prostate cancer, while the fifth lies 88 bp downstream of the annotated CpG island boundary (red circles in FIG. 3A). The two remaining CpGs showed either no differential methylation (gray circle in FIG. 3A) or slight but statistically significant hypomethylation (green circle in FIG. 3A); both lie further upstream of the transcriptional start site, outside of the promoter CpG island. Our data not only confirm the previously described hypermethylation of the GSTP1 promoter CpG island, but also show that CpG DNA methylation alteration is highly context dependent even within a single promoter.

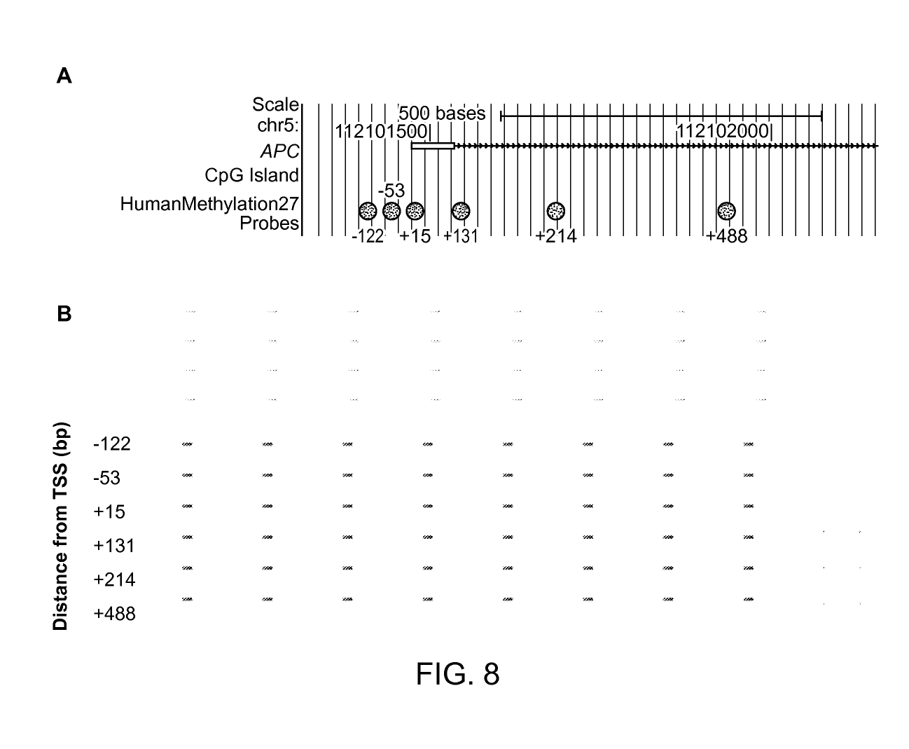

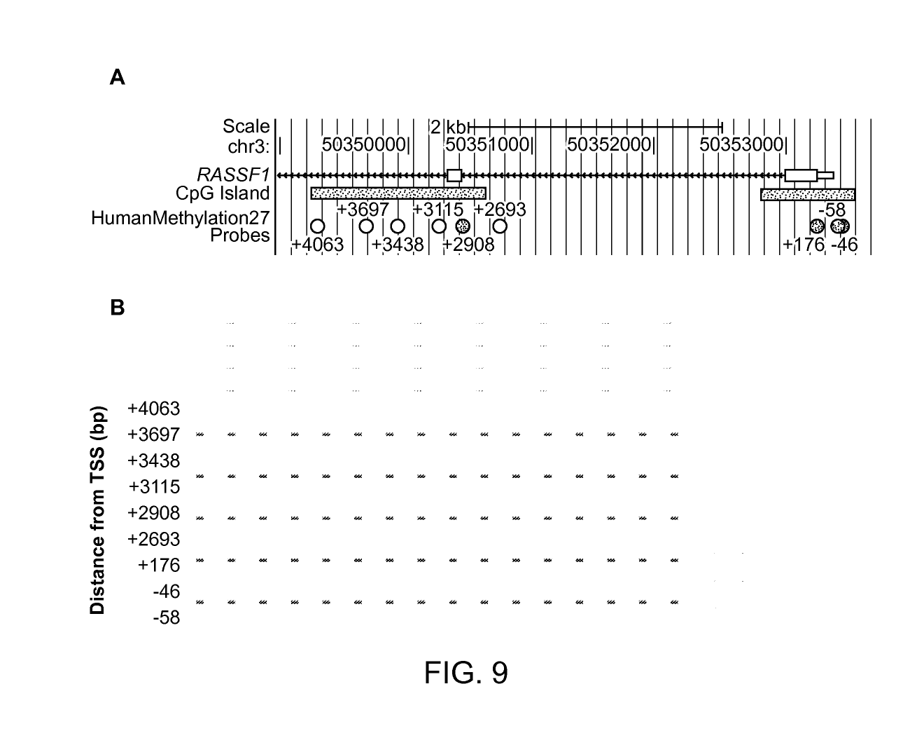

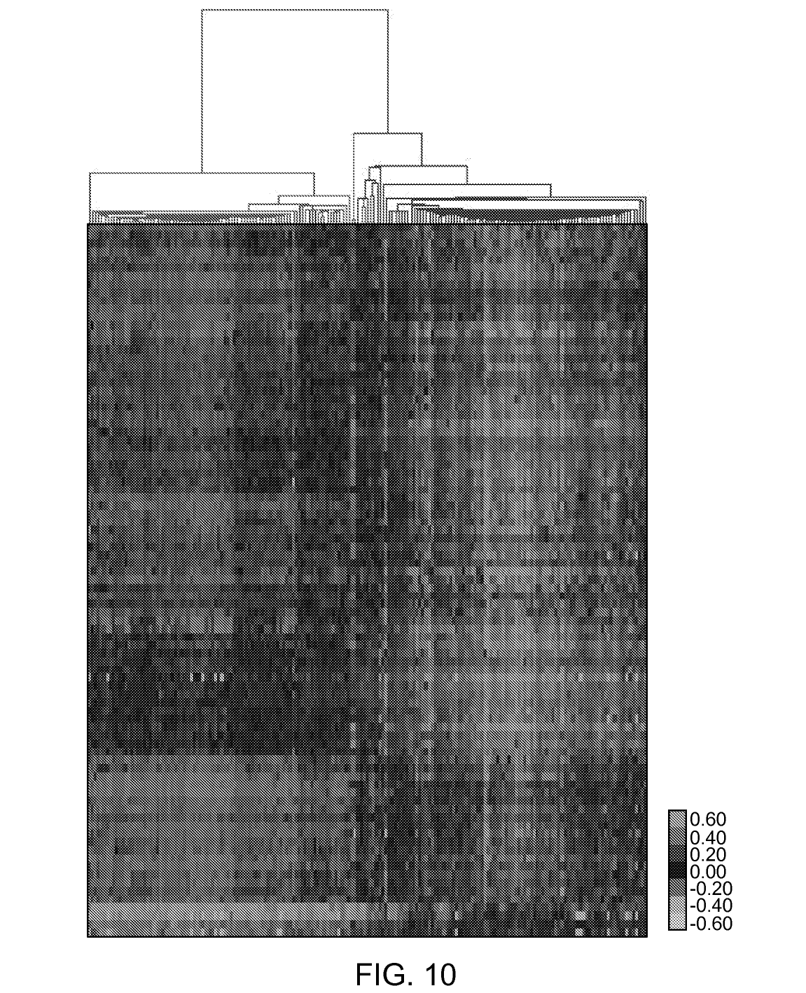

In addition to GSTP1, we also examined our data specifically for methylation changes in the promoters of APC and RASSF1, which have also been previously shown to have hypermethylation in prostate cancer and were represented by multiple probes on the HumanMethylation27 array. With APC, all six CpG sites represented on the array showed hypermethylation in tumors, located 122 bp upstream to 488 bp downstream of the TSS (FIG. 8). With RASSF1, three CpGs sites were probed, located 58 bp upstream to 176 bp downstream of the TSS and within a CpG island boundary; all three were hypermethylated (FIG. 10). However, five of the six probes located more than 2 kb downstream of the TSS in a second CpG island did not show differential methylation.

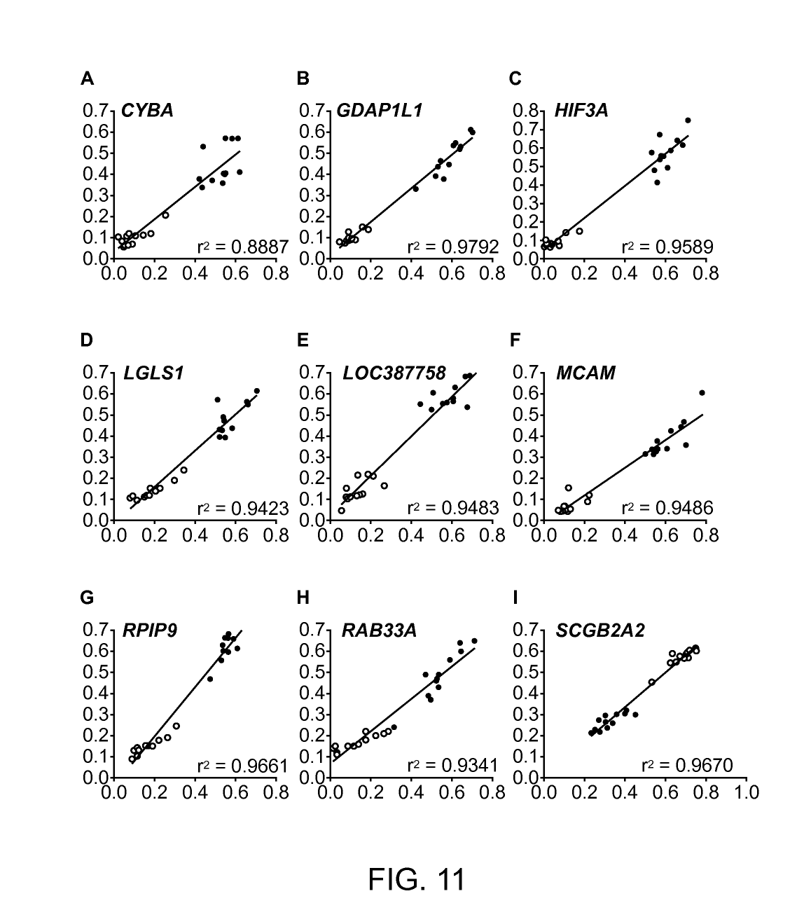

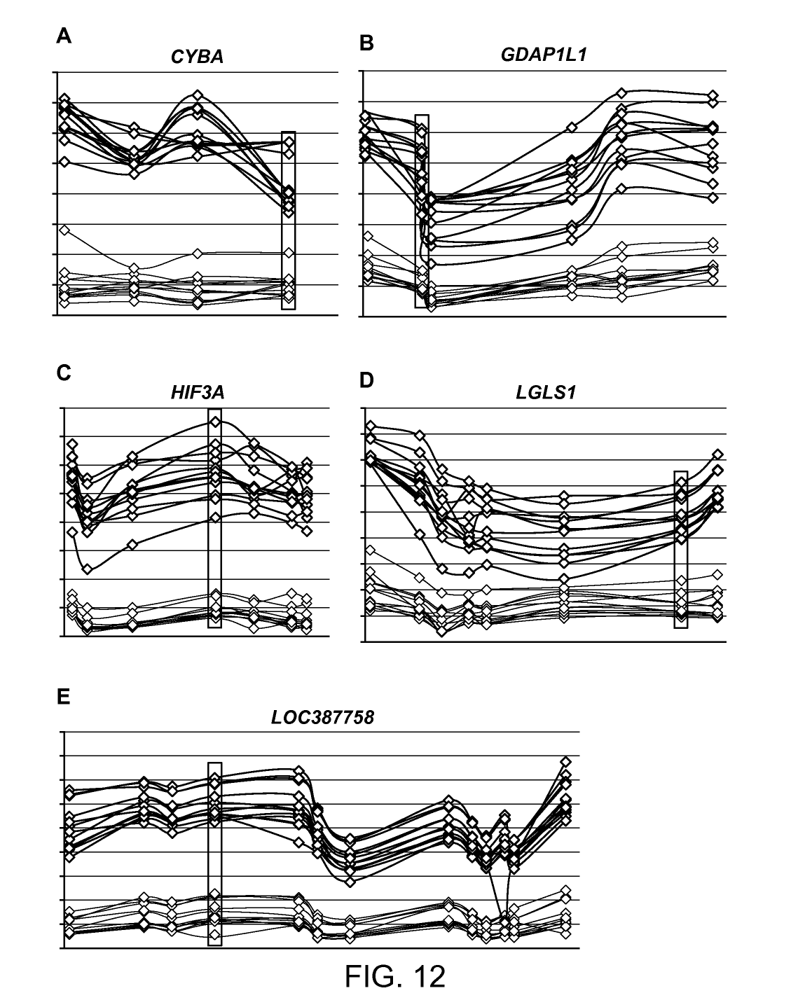

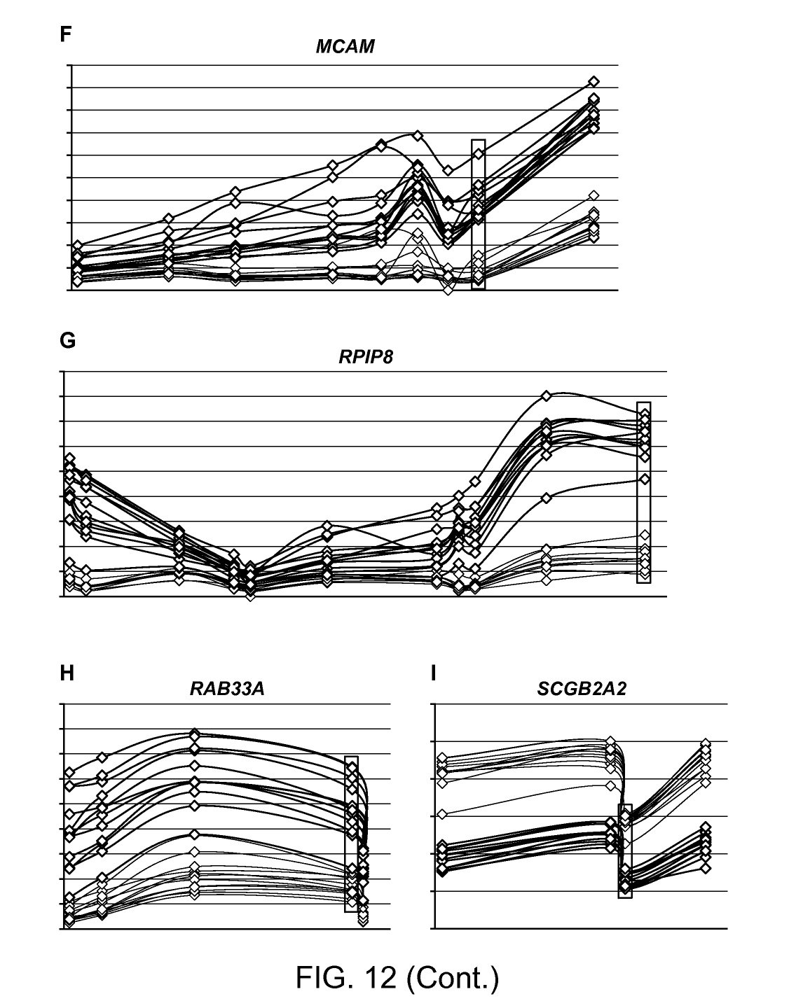

While hierarchical clustering of samples using the most differentially methylated CpG sites (the set shown in FIG. 2) was able to distinguish most tumors from benign adjacent tissues, the classification was not perfect, as indicated by the inclusion of benign adjacent tissue samples within the tumor cluster and vice versa. To identify CpG sites that could best predict either the tumor state or the benign adjacent state, we performed a Prediction Analysis of Microarrays (PAM), to perform sample classification. This analysis generated a list of 87 predictive CpG sites, most of which had increased methylation in the tumor samples (83/87), and represented 82 gene promoters total (FIG. 11, Table 1). The CYBA, GSTP1, KLK10, PPT2 and CXCL1 promoters each had two CpGs represented in this list. Notably, in this ranked list of 87 predictive methylation alterations, the GSTP1 hypermethylation was ranked 57th (Table 1). Thus we have identified 56 molecular events, most of which had not been previously characterized, that are better identifiers of prostate cancer than is GSTP1. We validated several of these diagnostic methylation markers by PyroMark sequencing.

TABLE 1

| CpG ID | SourceSeq | Accession | Symbol | SEQ ID NO |
|---|---|---|---|---|
| cg00489401 | GGGTGCAGGTGCACGCTGGCACTTGAGACGAATCTTGAGGAGGCGAATCG | NM_182925.1 | FLT4 | 1 |
| cg10541755 | CGCCCAGGGCGGGTGTCCCCACCCTCAGCGAGCTCCTCTGCGACTTCTCA | NM_020390.5 | EIF5A2 | 2 |
| cg05270634 | GGTGGTGGGAGACGCAGAGTGCGGCAAGACGGCGCTGCTGCAGGTGTTCG | NM_005440.3 | RND2 | 3 |
| cg02879662 | CGCCCCGGGGCGCGCAGTTGGAGGCACATCCCCACCGCACTCTCCACCCT | NM_022462.2 | HIF3A | 4 |
| cg17231524 | AGGTAGTTTCTGGAGCCCGATGGCAGGGGCCCATTCAGTGCGTTTCTGCG | NM_203306.1 | NCRNA00086 | 5 |
| cg26537639 | CGCCAGCGCCTGTTCGTTGGCCCACATGGCCCACTCGATCTGCCCCATGG | NM_000101.2 | CYBA | 6 |
| cg22262168 | TGACAGTTGTTGGCCCCAAAGTTAAGCGCGATTTGTACGGCCTTTACACG | NM_024761.3 | MOBKL2B | 7 |
| cg14563260 | CGCCTTCTGGAAAGTTTAGAAAGTGAGCCACGAAAGAGAGGCCACATTTC | NM_001401.3 | LPAR1 | 8 |
| cg19790294 | GATTTGCTCAGGAAGCCGACCTTCACACCTTGTCCTGCTATTAATAGACG | NM_000101.2 | CYBA | 9 |
| cg07186138 | GCATATCTAAGAGGCTGAACATGAATCCACAGATCAGGTACCTCTGCACG | NM_014508.2 | APOBEC3C | 10 |
| cg14672994 | ACTGGTGTTCCTTCTGAAGCTGACATCTGGCCTCAGCTGGGACTCTGGCG | NM_025149.3 | ACSF2 | 11 |
| cg21096399 | GGCTACATTGGCTGGCAGGGGCTGAGCAGCGGTGAGCCTGGCTGGCTTCG | NM_006500.2 | MCAM | 12 |
| cg15146752 | CGCCAGCGCCCCTACGGATTAGCCCCCAGGGATCTCTGAGCCTGGTATCC | NM_004431.2 | EPHA2 | 13 |
| cg24340926 | TGCGGCAGCCAATAGGAGCCGCTCTCCTGAACATTCAGAGGATGGGTGCG | NM_004794.2 | RAB33A | 14 |
| cg20557104 | CGGCACTGTGACTCCAGGAACACTCACATCCAGCCCCTTGGGGCAGGAGG | NM_198540.2 | B3GNT8 | 15 |
| cg04622802 | GAGGAGTTCCAGTCACCGAGCGAGGGGCGCAAGGGTGGGTGCATCCTGCG | NM_203371.1 | FIBIN | 16 |
| cg17965019 | TTTCAGAGCATAGCTTTCTCAACTATGGCCCGGACGAAGCAGACAGCTCG | NM_003535.2 | HIST1H3J | 17 |
| cg09300114 | CGGGTCATCACCCCAGGCCCCGGGGCAGCCCAGAACCAGGACAGGAAGAC | NM_004695.2 | SLC16A5 | 18 |
| cg08359956 | ACAGGACCGAGTCCTTGGCTGCCTGTGGAGCTCCTGTGCCAGCAGCTGCG | NM_014020.2 | TMEM176B | 19 |
| cg10453365 | ATCTTTTGGGGCCCTCGGCTTGGGTTGGGCCCCTGCCAGTTGGGCGAGCG | NM_016321.1 | RHCG | 20 |
| cg08924430 | TTTTAAAGACCCGACAAACTGGGAAATTGACCGAGTTCTGTTTCTCCCCG | NM_017628.2 | TET2 | 21 |
| cg13102585 | CGCTTTCTTGGAGGACAGCCCCAGAGCCATGGTGGTCTGGACAAAGCTCG | NM_006695.3 | RUNDC3A | 22 |
| cg00848728 | CGGACCTGCCAGCCCCAGGGAACAAAAGCGGAGCCCGCTCGCCCTCTACT | NM_021080.3 | DAB1 | 23 |
| cg03085312 | TGTGTATTTGAGACAGGGAACTGTTCCTGTCCCCAGCCGATGACCAGACG | NM_001024809.2 | RARA | 24 |
| cg06428055 | CGCAGACCTATGATGTGGAGAACTGGATCGCCAAAATAGAGACCTCTTGG | NM_001421.1 | ELF4 | 25 |
| cg04448487 | TTCCACCCTTGCAGGGAGCCTGACACTGAGGGCTGGCGGCTTTTCTGGCG | NM_024034.3 | GDAP1L1 | 26 |
| cg09851465 | CGGGCGTCCTTCTAGAAGCCCATCTCGCTCACCTGTGTGGTCACCCTTGT | NM_152377.1 | C1orf87 | 27 |
| cg08348496 | GCTCTAGCCGTCGAGGAGCTGCCTGGGGACGGTACGTGGCTTAGGGGTCG | NM_178232.2 | HAPLN3 | 28 |
| cg22862656 | AGGACCATCAGCAACTTCATGGTGAGGCTGCTGCTGTCGGTGTTCAGTCG | NM_002411.1 | SCGB2A2 | 29 |
| cg22319147 | CGCTCAGCCCTGGACGGACAGGCAGTCCAACGGAACAGAAACATCCCTCA | NM_001795.2 | CDH5 | 30 |
| cg27223047 | CGGGACCAAATTAGGGGCTGGGAGTTTCCAGATTGAAATGCGCCCTCCAC | NM_001999.3 | FBN2 | 31 |
| cg08965235 | GTCTCAAGGGCCAGTGTCGGGACAGTTGTCAGCAGGGCTCCAACATGACG | NM_021070.2 | LTBP3 | 32 |
| cg24715245 | CGGCCGGGCCCCCAAACCTTGCAGTCTCACTCGCCGGTGAGATAATCTGG | NM_004181.3 | UCHL1 | 33 |
| cg02254461 | CGACATGCCCCGGCAACCAAGTCCTGGCCTGGGAGCCCACCCTCAGCCCC | NM_033027.2 | CSRNP1 | 34 |
| cg26025891 | AAAAGGCATCTTTGAACTGCAGCTGGGGCATCATCCTCAGGCGTCTGCCG | NM_003978.2 | PSTPIP1 | 35 |
| cg01683883 | CGAGAGGAAGCAGGTGTTCTCGATAAAAGCAGCAGCCCTAATTTTATGGT | NM_144673.2 | CMTM2 | 36 |
| cg17606785 | CTCGGGGGCCTTTGGCTCCAGGCAACTTGGGGCAAGCGTCTCAGTTCTCG | NM_032459.1 | EFS | 37 |
| cg21307628 | CGAGCATGGAACTGAGAAAGTCCTGTATAGAGGTTAACTATAGAGTTGCC | NM_199511.1 | CCDC80 | 38 |
| cg18328334 | CGCCCCTGTCCTGGGAGTCCCTTGGCCCAGACACCCACCTGACTTAGTGG | NM_022648.3 | TNS1 | 39 |
| cg19853760 | CGCCTGCCCGGGAACATCCTCCTGGACTCAATCATGGCTTGTGTGAGTGT | NM_002305.2 | LGALS1 | 40 |
| cg16232979 | CGCCGATCGCCGACCCACATCCCTGCGCCCGCAGCCAGGACCCCCTACTT | NM_003290.1 | TPM4 | 41 |
| cg23502772 | GGATTACTGGGTCACGGTTTCCCAAGGACATGGAAACCCTTGCTGAAGCG | NM_153361.2 | MGC42105 | 42 |
| cg04034767 | TGGGGGCCCAGGGGTGGCGGCTGCGGCAGGGGGTCCCGGGGTCGGGACCG | NM_181711.1 | GRASP | 43 |
| cg20083676 | CGCCCGCCCAAGCCCAGACCTCGGACCTGGTTCCAAGCCTGTTCCCGCTG | NM_005226.2 | S1PR3 | 44 |
| cg21623671 | CGGGTCCTTTGACTGGCGTCCAGCTGACCCCAACCCCGGACCTTCAAAGT | NM_001155.3 | ANXA6 | 45 |
| cg12627583 | CTTTCTCCGTCGGGGTGGATGGGTTGGACTTTAGGCTCCAGCAAGCCCCG | NM_001159.3 | AOX1 | 46 |
| cg19713460 | CGGCGCCTACTGCGTACCAAGCACCCTCTAAGAAGGACGAACACAGCTCC | NM_145738.1 | SYNGR1 | 47 |
| cg19423196 | TTTCTCACATGATTTTTCAGGCACTTTCGCTTTTCCATATATAGGAGTCG | NM_000429.2 | MAT1A | 48 |
| cg22892110 | CGCACCCCCAGGCACTCACCCCCTGCCCGAGCTGCCGCCTGAGTAGGTAT | NM_139021.1 | MAPK15 | 49 |
| cg12727795 | CGCCAGCCGCCAGCTGCTGAGTCACTTTTGTCAAAGAGTGGCCTCGGCCC | NM_002609.3 | PDGFRB | 50 |
| cg15835232 | CGCCCCGGCCCCGCGCTGATGAAATTGAGGAGCTCACCCAGCACCCTTCC | NM_002126.3 | HLF | 51 |
| cg12100791 | CGGGGTTCTAGAAATCCGAGGTTCTAAGCCTAGGTGCTCCAATAAACCCA | NM_013258.3 | PYCARD | 52 |
| cg09704415 | CGGGGGCAATAATTCTCTAAGAGAACTGGAGCCCGAAAGAGGAATGAAAA | NM_019073.1 | SPATA6 | 53 |
| cg04337944 | CGCCCTGGGCTCGGTAACCCCCAGCCAGCGTCCCCCAGCCCAGCTAGCGC | NM_001996.2 | FBLN1 | 54 |
| cg14360917 | CGACAGCAGGACCAGCTGTCCTCACAGCCTCAGATGGCTGAGTCTGAGGA | NM_003110.4 | SP2 | 55 |
| cg26420196 | CGGCCCCGCTCGATTCCTGGAATCTTATTTTTGGACCTGCTGCCGCAAGC | NM_000820.1 | GAS6 | 56 |
| cg04920951 | CGGCCTCCGAGCCTTATAAGGGTGGTCCCGCCCCGCTCCGCCCCAGTGCT | | GSTP1 | 57 |
| cg27554782 | TGTCTGTGGGCTGGGCAGTGGGCTGGATGACACCGGCTTTGCAGGCACCG | NM_000750.2 | CHRNB4 | 58 |
| cg00727590 | GTATGGGGTGATCTTGGGCTTGTAACCGAATCCACCAGCCGGGCAGGACG | NM_015715.2 | PLA2G3 | 59 |
| cg14188232 | CGCCCAGGAGGGCCACCAGATCTGGGAGCTTTTCAACTCAAGCCTCTTCA | NM_001004439.1 | ITGA11 | 60 |
| cg18145505 | CGGGAGGAGAATAAAACTAAATGACCGTCAAAAGTCAAGGCTTCTGTTCC | NM_013372.5 | GREM1 | 61 |
| cg18711066 | GATGGCTGGCTCTAGGGAAGGCATCAGGGCCCCTCAGAGTTACCTGGACG | NM_004555.2 | NFATC3 | 61 |
| cg26124016 | CCTTTACGCCTTTTTATTTGCGGCGGCTTAGCTTGGAAAACGGTGTTCCG | NM_000965.2 | RARB | 62 |
| cg24512400 | ACGGGAAGATGCCGCGAGGGGCGTCATTAGGGTAATTGTGCCCATTACCG | NM_002776.3 | KLK10 | 63 |
| cg15528736 | CGGGAACCACAGAGAAGGAAAAAGAAGAACCACAAGCGTTTTGAGAAACA | NM_004107.3 | FCGRT | 64 |
| cg01777397 | CTAATTGCTCAACGTGGGTGTAGCACGGATTAGGCCTTTTACAGCAAGCG | NM_024692.3 | CLIP4 | 65 |
| cg03513363 | TGGACTTTGATCGCCGAGGGCTCTCTGCTCTTCAGAGTCTGCTTGGAACG | NM_080611.3 | DUSP15 | 66 |
| cg21790626 | CGCCTTCGTGGCCCCAACTCGGCGCTCTGCTATCTCTGATCCGGTGAACA | NM_003444.1 | ZNF154 | 67 |
| cg02659086 | CGGGGGGAAATTCCCTAAGACCGCTGCGATCCCGGAGCTTGCACACCCGC | | GSTP1 | 68 |
| cg00862041 | GGTTGGGGCGGGGGTGCAGACACATCACGGGGCGGTTTGGTATCCATCCG | NM_138437.3 | GPRASP2 | 69 |
| cg18552413 | CGCCCACTGCCTGCACAAGCCTCAGGCCTATGGGGGTCACTGGCCTTGGG | NM_002036.2 | DARC | 70 |
| cg23499956 | CGACCCAAGCAGACCCCACTGTGTTCCAGGAGCTGTTCCTTGAGAGGGAT | NM_080388.1 | S100A16 | 71 |
| cg17329164 | GTTGAAAGACTGGTCGAAATTACGCGGGCATGAGTCAGCGCATCCCTACG | NM_005155.5 | PPT2 | 72 |
| cg18006568 | CTGCTTGGACTCCGCGTCAGTCCAGGTGGCCTTCAAGGAGACTTTGTTCG | NM_024933.2 | ANKRD53 | 73 |
| cg14539231 | CGCCGAGCCCGGAGTTCACCACTCTATTGCGGGTGTTCATGGTTCACAGC | NM_001002264.1 | EPSTI1 | 74 |
| cg04273431 | CGGAGAACTGTGGCATCCCAGGCCCACCGTCTTCACCAGTAGCAGCCCGC | NM_025263.1 | PRR3 | 75 |
| cg15910208 | GGGAGATTCGGGCTGGAACAGCGGTAATGGGCACAATTACCCTAATGACG | NM_002776.3 | KLK10 | 76 |
| cg12585943 | TTACAACTCTTCATTCTGAAGTGCGTGTAGTGCCCTTGTCTCCAGAGACG | NM_005155.5 | PPT2 | 77 |
| cg15309006 | AGGGAGGCTGCGAACAACGGGCTGTTTCAGCTCCGAGATTTTGCGATCCG | NM_022097.1 | CHP2 | 78 |
| cg17568996 | CGACAACCAGCAAATCCCCAGAGACAGGTCCCTGGGAATTAGCTGCGCCG | NM_145912.4 | NFAM1 | 79 |
| cg24467291 | TCAAACGAACGGAGCAAACCCTGGGATCGTTTCAAAGGATTTTTAACCCG | NM_002956.2 | CLIP1 | 80 |
| cg02029926 | CCTCGCCCTTCAGAGTAACTCCTGTGGACTCTGAGACTCTGGGATATTCG | NM_001511.1 | CXCL1 | 81 |
| cg20786074 | CGCACAGCTTTGTTTAAAAGTCCCAGGTTGTGTGGAGGGGCAGCCCAAAG | NM_018894.1 | EFEMP1 | 82 |
| cg25806808 | AATATCCCAGAGTCTCAGAGTCCACAGGAGTTACTCTGAAGGGCGAGGCG | NM_001511.1 | CXCL1 | 83 |
| cg23092823 | CGGACTCCAGACCGCCAGCTGAGACCTTTAGCTCAACTAGTGGTTGGCAC | NM_153703.3 | PODN | 84 |
| cg09099744 | CCTACCGGCATTGAAATACTTATGGATAAAGTTCTCGCAATGGCTTCACG | | CDKN2A | 85 |
| cg25259754 | CGGCCTCAGTTCCTAAAGGTGACCAGGGAAAAACTCAAGGAGCTTCTATC | NM_052939.3 | FCRL3 | 86 |

Table 1. Diagnostic methylation markers of prostate cancer identified by PAM. CpG ID: Designated by Illumina. Chr/Mapinfo: chromosome number and coordinates based on NCBI36/hg18. SourceSequence: sequence upstream of the CpG. Gene ID/GID/Accession/Symbol/Gene Strand/TSS Coordinate: annotation of nearest gene provided by Illumina. This list of diagnostic markers include both hypermethylated and hypomethylated promoter regions. While the vast majority showed hypermethylation, 4 were hypomethylated: FCRL3, DARC, SCGB2A2, URB. In other words, most of these sites have low methylation in normal prostates, but high in tumor. However, those 4 sites have high methylation in normal prostate, and low in tumors.

Prognostic methylation markers. To explore tumor heterogeneity, we compared the methylation profiles of the 86 tumors with respect to Gleason grade and time to biochemical recurrence (defined as serum PSA>0.07 ng/mL after surgery) of the donors. Gleason grade is a powerful predictor of treatment failure, tumor progression and death from prostate cancer, and biochemical recurrence has also been correlated with prostate cancer-specific mortality. Next, we conducted a SAM survival analysis with the time to biochemical recurrence as the survival variable. With a false discovery rate of 26.8%, we identified six CpGs that showed greater methylation in tumors from men who had shorter time to recurrence and 63 CpGs that showed lower methylation in patients with shorter time to recurrence (Table 2). This strong bias towards lower methylation in aggressive tumors was striking as we observed a bias for CpG sites with increased methylation in the tumor/benign adjacent comparison. At a false discovery rate of 26.8%, we expect that 18 of those calls to be false. At a lower false discovery rate cutoff of 1%, we only observed four CpGs that showed higher methylation in patients with shorter time to recurrence and none that showed lower methylation (Table 2). While we were only able to identify a small number of CpGs whose methylation state correlated with time to recurrence, we noted that several of these CpG sites are in the proximal promoter genes of known cancer-related genes, including 3 CpGs near MAGE gene family members which encode for strictly tumor-specific antigens (Chomez et al. 2001) and 4 CpGs near WT1, a transcription factor gene associated with Wilm's tumor.

TABLE 2

| | | | | Direction of | SEQ |
|---|---|---|---|---|---|
| CpG ID | SourceSeq | Accession | Symbol | Change | ID NO |
| cg01352108 | CGGACAGGCACCACGCTAATCTGGCATCTCCCAGGCCCATTACCGGATCG | NM_016611.2 | KCNK4 | Hypermethylated | 87 |
| cg24068372 | GTGGCAGAGGCCAGAGCCCAGAGGCGCAGCCCGGGCAGCTAGGAGGGTCG | NM_198285.1 | LOC349136 | Hypermethylated | 88 |
| cg20870559 | GGAACTGTTCGGGTTCCTGCAGGACGTCACAGATGGTGTTCACCATCTCG | NM_002535.2 | OAS2 | Hypermethylated | 89 |
| cg03734874 | CGGGCCTCACACAGGCCGACTCTGGGTCGTCAGTTCCTCATCAGCTCGAA | NM_207379.1 | FLJ42486 | Hypermethylated | 90 |
| cg03640944 | CGCCCAACCACCGAGTGGTGGAGCCAGGACTCAACTCAAGTCTGCCCCAC | NM_033397.2 | KIAA1754 | Hypermethylated | 91 |
| cg02320454 | GGTCAGGTTGAGACCCCAGCCCAGCAAGATGGGCACGGAAATGTTGGGCG | NM_199243.1 | GPR150 | Hypermethylated | 92 |
| cg17173423 | TTGCGGGCTGACTGACCAGTGTGCTAATCACATCTGCATTTGGGGCCTCG | NM_006138.4 | MS4A3 | Hypomethylated | 93 |
| cg05047411 | CCAGGCTCTGCCAGATCTCAAAGTGAGAACCTTGAGGGATGACTGAACCG | NM_005364.3 | MAGEA8 | Hypomethylated | 94 |
| cg26164184 | CGGAACCCGAGGGGGGTCTTAACTAGTCATAGTCTCAGGACCACACATCT | NM_004108.2 | FCN2 | Hypomethylated | 95 |
| cg04645174 | GCCTGCGAAGGCATCTCAGTATGTGTAATGCATCCCCTCTTTTTTTCCCG | NM_030901.1 | OR7A17 | Hypomethylated | 96 |
| cg05828624 | CGGGAAGATACAGCATGAGTTTCTGTCCAAGAGGTTTTAGCTGTAATGAA | NM_002909.3 | REG1A | Hypomethylated | 97 |
| cg21325760 | CGCCGCCGCCCATCCGACCTGCCCCACAGGTCCTGGCCACCCAGCCACCG | NM_019066.2 | MAGEL2 | Hypomethylated | 98 |
| cg20804821 | CGACATTGGGGTGGGGAACCCTGACATTCACTGATTAGTCAAGACTGGGT | NM_080865.2 | GPR62 | Hypomethylated | 99 |
| cg03600318 | CGCAGGTGGGGATAAGAGTGAGTGAGTCAATAAAGAAGAAAATTGCCCCA | NM_003019.4 | SFTPD | Hypomethylated | 100 |
| cg11061975 | TAATGACCTACTGGGTTTCAGGCATGATATGCTCATTACCTCTTTAATCG | NM_018556.2 | SIRPB2 | Hypomethylated | 101 |
| cg14620221 | AATGACAATGGCTGCTGAGAATTCCTCCTTCGTGACACAGTTTATCCTCG | NM_012378.1 | OR8B8 | Hypomethylated | 102 |
| cg13311440 | CGGGATGTAGTTCAACCCTAGAAGCCAGATCTGGTGTCTGGAAAGCAGGT | NM_001778.2 | CD48 | Hypomethylated | 103 |
| cg27504299 | CGCCTTCACCAGATACCTCCAGGGGCAAGAGTCCACTGAGGTTACAGCGC | NM_004918.2 | TCL1B | Hypomethylated | 104 |
| cg03109316 | CGGGGGCATGCAAACCACAGTTGACCTACTAGCTGAAGCAGTGATAAAAG | NM_007136.1 | ZNF80 | Hypomethylated | 105 |
| cg00918005 | CGCAGACACTATGCTGCCTCCCATGGCCCTGCCCAGTGTGTCCTGGATGC | NM_001008387.1 | REG3G | Hypomethylated | 106 |
| cg17836145 | CGGCCCACCCAGAAAGTGAAATCAAAACAGGAAGTCACCAGGGGTGACTG | NM_004665.2 | VNN2 | Hypomethylated | 107 |
| cg15457079 | CGTGGGATGGATCAAAAGGGACAGAGAACTCTTTTTGAAAGTTGTAATAA | NM_001308.1 | CPN1 | Hypomethylated | 108 |
| cg07688234 | CCCTATGTGGAAAATGCATAATCTCTAACATAATGACGGGGTCAACCTCG | NM_002621.1 | PFC | Hypomethylated | 109 |
| cg22511262 | CGGAGCCCCTGTAGTTTGCCCTCTTCATTTATTTTCAGTGGATTTCCACG | NM_009237.17 | WT1 | Hypomethylated | 110 |
| cg24169915 | CGTGCAGAACCTGTGTTTACAGCCATGATAATGCATCTTGGGGGTTCCTG | NM_182560.1 | FLJ25773 | Hypomethylated | 111 |
| cg03833774 | TTGAATGTGTGCTTTCCACAGTTTCTGATCCTCAGCTCCCACTCTCTTCG | NM_152694.1 | ZCCHC5 | Hypomethylated | 112 |
| cg20832020 | CAGAAGAGGCCACATCTGCTTCCTGTAGGCCCTCTGGGCAGAAGCATGCG | NM_173799.2 | VSIG9 | Hypomethylated | 113 |
| cg17338403 | CGCTGCAGTTGAGAACTAGCAGATCCTATTGGTAGTGCCCTGTGGCCCAC | NM_013272.2 | SLCO3A1 | Hypomethylated | 114 |
| cg01564343 | CGTGTTATTGTGAATGCCACACCCATACCAGCAGCTGGGCTGGGAGATGC | NM_178174.2 | TREML1 | Hypomethylated | 115 |
| cg22228134 | CGATCTTTGCTGAGTGTCTATCTAGCCTCAGATTTATAAGTCTGGGTGTG | NM_033423.2 | GZMH | Hypomethylated | 116 |
| cg22442090 | CGTGCCAGACAGCTTACCAGGGTCAGTCACGAGCCCAGAGTCAAACCCTG | NM_018384.3 | GIMAP5 | Hypomethylated | 117 |
| cg01731341 | GTCACGTGGAATCATCTAAGTGGTGAGCAGCATTTCTGCCCCCTTTATCG | NM_020996.1 | FGF6 | Hypomethylated | 118 |
| cg19000186 | CCTGTTTTCGCCTCAATGTTGCATTTTCTGAGACCACTCTAGCTGTCACG | NM_000087.2 | CNGA1 | Hypomethylated | 119 |
| cg15711744 | CTTTACAGTGTATCCTAAATCTGATCACTTCTCATCATCTCTAGGCCACG | NM_012404.2 | ANP32D | Hypomethylated | 120 |
| cg03544379 | AGACCTTGACCATTTTGAGGCTGTCCTTCTGCACAAATATGGAAATTCCG | NM_012377.1 | OR7C2 | Hypomethylated | 121 |
| cg07443748 | CGGACCAGCACTCCACTGTGGGTCCAAGGATGAGCTCCAAAGAGCCCAGT | NM_014406.4 | CESK1 | Hypomethylated | 122 |
| cg04353769 | CGGTGATGGTTCAGGTTGTGTTTCTGGGTTCATTCTGGAAGCTCCCCCAA | NM_022349.2 | MS4A6A | Hypomethylated | 123 |
| cg04014889 | GTCTCCGGTGTGGCAGGCAGGTTTTTCCAGGCAGCTGGCAGGTGTGCTCG | NM_019066.2 | MAGEL2 | Hypomethylated | 124 |
| cg07379574 | AGTGCTTGGGGATGCAGGTCCTTGCGATAAGGGGCCGATACCACCTCCCG | NM_012109.1 | C19orf4 | Hypomethylated | 125 |
| cg10994126 | TTTGCTCCTTAATCACTGTCACAGACAATTGATACTGCCATTGATACTCG | NM_020318.1 | PAPPA2 | Hypomethylated | 126 |
| cg03014957 | CGGGGAGACACACAGATAAGTAGACCATTCAAAAGTAGGTTTATGCTAGA | NM_054112.1 | DEFB118 | Hypomethylated | 127 |
| cg24012708 | CATTTCCTGGACAAACTTCCTCCAAGGCTCCCCCAGATTTACCAGTGACG | NM_031219.2 | HDHD3 | Hypomethylated | 128 |
| cg13447818 | ACTAGCCTCTCTCTCTACTATTAAGCTGGCTTACCATCTTATGTCATTCG | NM_002016.1 | FLG | Hypomethylated | 129 |
| cg05222924 | CGAGTTTTATACTTAATTTGCCAGGGGTTCGCTGCAGAAGCGGCAGAGAC | NT_009237.17 | WT1 | Hypomethylated | 130 |
| cg18368125 | CACATAGATATTCATCATAGAACTGCCATGATACTCCCATGTTTGGCTCG | NM_144676.1 | TMED6 | Hypomethylated | 131 |
| cg19718882 | GTGTGTGCGGGCCCAGGACTTACTCGAAGGGCGCACTTCTTGGGAATGCG | NM_015855.2 | WIT-1 | Hypomethylated | 132 |
| cg13097816 | CGGGCTAGAGTCATCCTGACTCGGCCACCCCTGCAGCTGGGCAAACTTGT | NM_005301.2 | GPR35 | Hypomethylated | 133 |
| cg12237269 | TACCTGAAGAGGATCAAAGACACACCCTGGCTATGGCAGGTTTCTCCTCG | NM_003063.1 | SLN | Hypomethylated | 134 |
| cg19241311 | CGAAGCTTTGTGAAGATCACAGCTACCTTAATGGGAGAGAAAGCTCATTT | NM_153324.2 | DEFB123 | Hypomethylated | 135 |
| cg16777782 | CGCCCGGCCTATCGTGCCCTTTCAACAGATGAAGAAACTGGTGAGTTTAA | NM_010498.15 | CDH13 | Hypomethylated | 136 |
| cg05248470 | CGAGTGGGATTCATGACAACAATCTGCAAAGGAAGAAACTGAGGCTCAGT | NM_005874.1 | LILRB2 | Hypomethylated | 137 |
| cg16158220 | ATCAGAGCCTCCTAAATCTGTTCATGTCACACTGTCAGGTTTGGGCTACG | XR_000606.1 | REGL | Hypomethylated | 138 |
| cg21353232 | CTGGTGACAGCTGTGAATCTACTAGAACACTACACATAGCCACAAAATCG | NM_021115.3 | SEZ6L | Hypomethylated | 139 |
| cg13482233 | TACTGGTGCTTCCACCTGCCTTGGTCTGAGTTGCAGTCCATGGGGCAGCG | NM_014799.2 | HEPH | Hypomethylated | 140 |
| cg12234947 | AGACATGCAGAATCTCAGGCTTTAATCCAGAACTTCTGATTCAGAATCCG | NM_005272.2 | GNAT2 | Hypomethylated | 141 |
| cg15075718 | CCATGAAGGACTTCTCAGATGTCATCCTCTGCATGGAGGCAACAGAATCG | NM_031433.1 | MFRP | Hypomethylated | 142 |
| cg01351032 | CCTTGGGGCTCTGACAGGTAGGACCCAGCAGGGCGTGGAGCCAGGCAACG | NM_000246.2 | CIITA | Hypomethylated | 143 |
| cg01693350 | CGGCACCCACTCTCGAGACGTCCGTCCGCACCCCAGAACTCGGGCCCAAG | NT_009237.17 | WT1 | Hypomethylated | 144 |
| cg12878228 | CGGTGGACAAAATGGGAAAAGCTCAGAAACTTGGTGTTGAAATCGGACCT | NM_002769.2 | PRSS1 | Hypomethylated | 145 |
| cg06550629 | CGCACCTGCGCACAAAAGACCACAGTGTGAGACACACTCAGGGAAAGCCT | NM_198827.2 | GPR133 | Hypomethylated | 146 |
| cg01757745 | TTTCCCTAGGTTGGCCGATTTGATCAACTCGTAGGCCTTCTTCAAGGACG | NM_173572.1 | C10orf93 | Hypomethylated | 147 |
| cg02813121 | AGAAACCTGCCCAAAATGGATTAAGTCTCATCTGTACATTCCCCATGTCG | NM_005621.1 | S100Al2 | Hypomethylated | 148 |
| cg06806711 | CGCTGATAGACATCAGGTGACAGGAAATCAGTAGCTTCTGCTACCTTGGG | NM_152866.2 | MS4A1 | Hypomethylated | 149 |
| cg13297249 | TGCAGACGTCCCCACAGAGGGCAGTGCCGAGGACAGTGTGTGTGCAGACG | XR_001026.1 | FLJ38379 | Hypomethylated | 150 |
| cg01369413 | ATGGCACGGTGCTGCCTCTTGATGACCAGGTGGACAGTGAGGCCATCTCG | NM_017481.2 | UBQLN3 | Hypomethylated | 151 |
| cg09217923 | TGATCTCAACCACACATGGATGGGACCTCTGGTTCAAGCAGAAGAATGCG | NM_001033080.1 | TAAR2 | Hypomethylated | 152 |
| cg00690280 | CGTTCTGCACTGATTCATTGTGTGGTCTTGAGCAAGTTGTAGAGCTTCTC | NM_172006.2 | WFDC10B | Hypomethylated | 153 |
| cg21742836 | CGGAAGCAACTAAGAAATGTCAAGAGTGCCATTTTGGAATCAGAGAAGTC | NM_002720.1 | PPP4C | Hypomethylated | 154 |
| cg24122922 | CGCCCCCGTTTCAACACTAGGCAGAGGCCCCAGTCCTGCCACCCGCAGGC | NM_024893.1 | C20orf39 | Hypomethylated | 155 |

Table 2 CpG sites with methylation patterns that correlated with time-to-recurrence after radical prostatectomy identified by SAM. CpG ID: Designated by Illumina. Chr/MapInfo: chromosome number and coordinates based on NCBI36/hg18. SourceSequence: sequence upstream of the CpG. Gene_ID/GID/Accession/Symbol/Gene_Strand/TSS_Coordinate: annotation of nearest gene provided by Illumina.q-value (%): indicates lowest FDR at which the site is called significant.

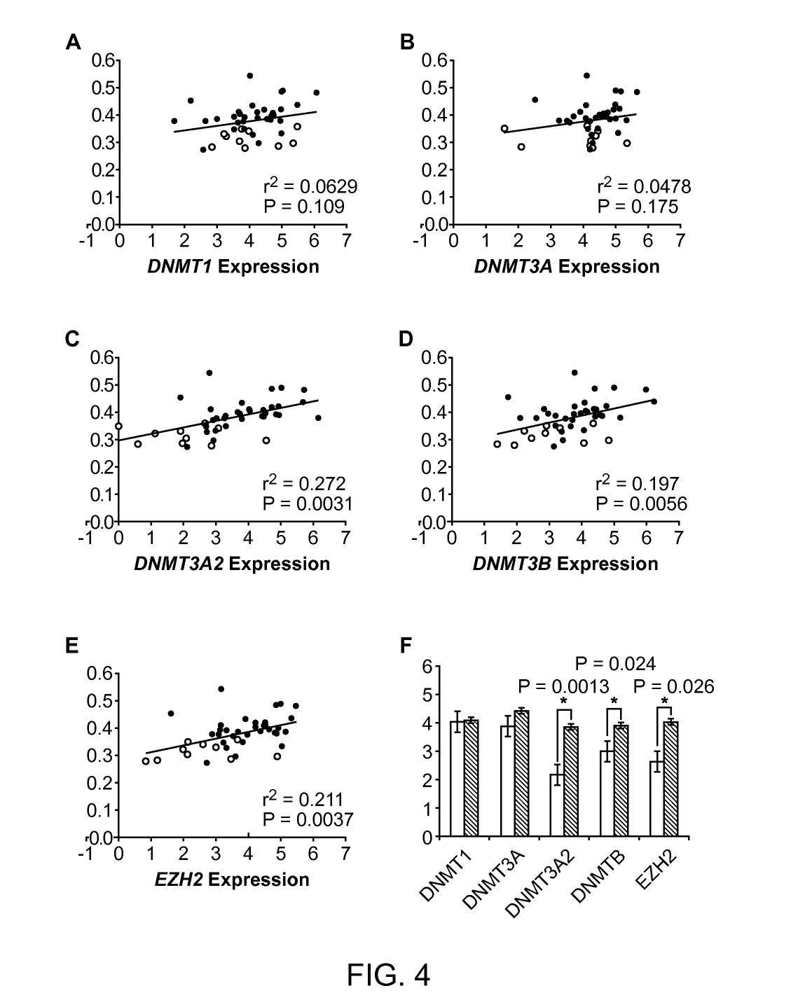

Correlation of tumor hypermethylation with DNA methyltransferase expression. With nearly one third of assayed CpGs showing changes in DNA methylation between tumor and benign adjacent samples, we hypothesized that one or more of the DNA methyltransferases (DNMTs), or a protein that interacts with a DNMT, had altered activity, possibly due to changes in transcript abundance, in prostate tumors. Such alterations in activity could in turn lead to global DNA methylation changes. To test this hypothesis, we selected RNA from 10 of the benign adjacent and 36 of the tumor samples, and measured the transcript abundance of DNMT1, DNMT3A, DNMT3A2, DNMT3B, DNMT3L and EZH2 using the TaqMan Gene Expression assay. These genes comprise the known maintenance methyltransferase (DNMT1), all known methyltransferases with de novo capability [DNMT1, DNMT3A, DNMT3B], and two interacting proteins thought to target methyltransferases to specific genomic regions [DNMT3L and EZH2]. In addition, we uniquely assayed DNMT3A and its alternative promoter variant DNMT3A2 by using transcript-specific primers and probes. While several splice variants of DNMT3B have been characterized, we were unable to design variant-specific primers and probes for them, so instead we designed primers and probes to the common region of all DNMT3B variants. We did not observe detectable levels of DNMT3L transcript abundance from either tumor or benign adjacent samples. When the transcript levels of the remaining genes were compared between benign adjacent and tumor samples with a two-tailed t-test, three showed significant changes: DNMT3A2 (P=0.0013), DNMT3B (P=0.024) and EZH2 (P=0.026), while DNMT1 and DNMT3A did not (FIG. 4F).

We compared the expression values for these five genes to global DNA methylation levels. Specifically, we plotted the mean percent methylation of all 5,912 hypermethylated CpG sites against relative expression of each methyltransferase or interacting protein, and calculated regression and the goodness-of-fit of the regression for each sample. Again, DNMT3A2 (r²=0.272, P=0.0031), DNMT3B (r²=0.197, P=0.0056) and EZH2 (r²=0.211, P=0.0037) all showed significant correlation between expression and global hypermethylation, while DNMT1 and DNMT3A did not (FIG. 4A-4E). The correlation between DNMT3A2, DNMT3B and EZH2 expression and global hypermethylation, in conjunction with the observed over-expression of the same genes in tumors, suggests a causal role in the global methylation changes seen in prostate tumor.

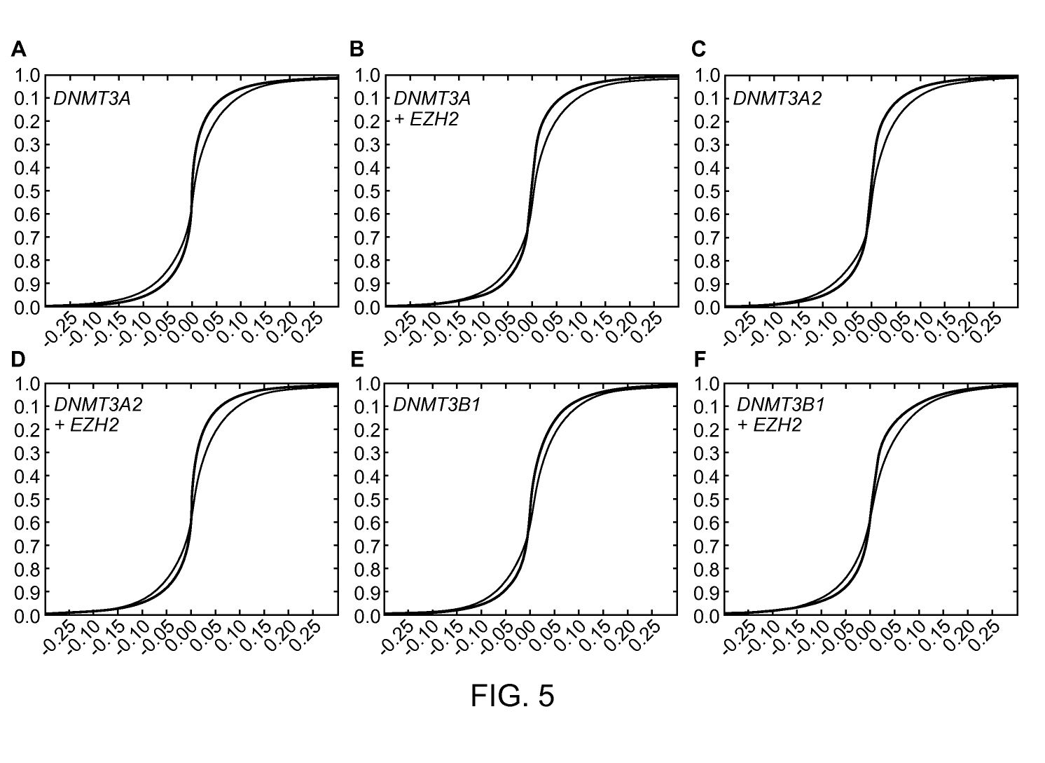

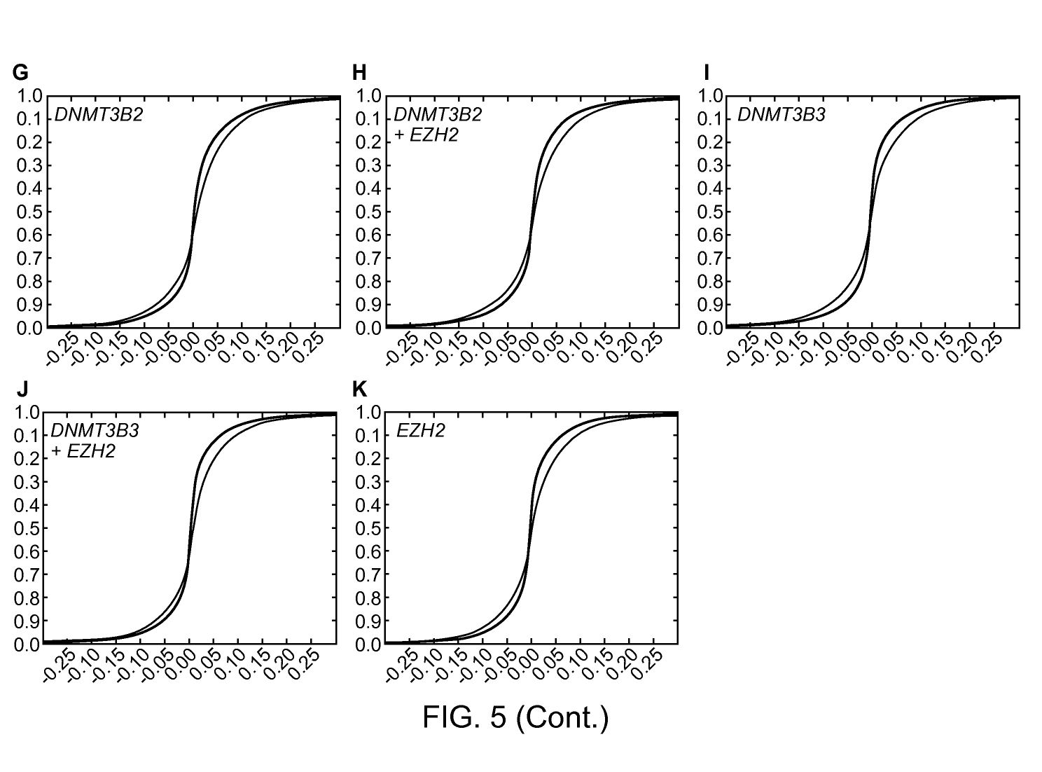

DNMT overexpression recapitulates hypermethylation events seen in prostate tumors. To determine whether the increased transcript abundance of DNMT3A2, DNMT3B and EZH2 in tumor cells has a causal role in the hypermethylation of a large number of promoter CpGs, we expressed these genes from the CMV promoter in transient transfection assays in primary cultures of normal prostatic epithelial cells. We used plasmids expressing DNMT3A, DNMT3A2, DNMT3B1, DNMT3B2, and DNMT3B3, an EZH2-cDNA plasmid, and a no-insert plasmid. We co-transfected each cDNA plasmid with the no-insert plasmid, and independently with the EZH2 plasmid, and also included a mock no-insert plasmid only transfection. We calculated the change in DNA methylation for each CpG between each cDNA transfection and the mock transfection after 48 hours. We then plotted the ideal cumulative distribution function of the DNA methylation level change at all 26,333 CpG sites along with the empirical cumulative distribution function of just the changes at the 5,912 CpG sites hypermethylated in tumors (FIG. 5A-5K), and tested the difference in the two distribution functions using the Kolmogorov-Smirnov (K-S) test. In all eleven experimental transfections, the distribution of the 5,912 CpG sites was significantly enriched compared to the null: DNMT3A (P=6.0E-45), DNMT3A2 (P=3.5E-62), DNMT3B1 (P=1.2E-31), DNMT3B2 (P=5.2E-39), DNMT3B3 (P=4.6E-44), EZH2 (P=1.1E-59), DNMT3A+EZH2 (P=7.8E-64), DNMT3A2+EZH2 (P=9.8E-65), DNMT3B1+EZH2 (P=2.1E-29), DNMT3B2+EZH2 (P=6.7E-42), DNMT3B3+EZH2 (P=2.5E-67). Consistent with our hypothesis, when the plots of the empirical cumulative distribution functions were visually inspected, we observed that the low P-value of the K-S test appeared to be driven more by the CpGs of increased methylation rather than CpGs of decreased methylation in all eleven conditions.

To test specifically whether the list of 5,912 CpG sites was statistically enriched for CpGs with substantially increased DNA methylation, we performed a series of chi-square tests. Based on the distribution of CpG methylation levels in tumor and benign adjacent tissues at these CpG sites, we set a cutoff value of 0.05. In other words, CpG sites where the methylation increased by 5 percent or greater in the experimental transfection compared to the mock transfection were considered to have substantially increased DNA methylation. We calculated expected values based on the distribution of these CpGs with substantially increased DNA methylation in the entire set of 26,333 CpGs. When chi-square tests were performed, all eleven experimental conditions had very low p-values: DNMT3A (P=1.1E-45), DNMT3A2 (P=1.7E-66), DNMT3B1 (P=8.9E-127), DNMT3B2 (P=1.8E-157), DNMT3B3 (P=6.6E-10), EZH2 (P=9.4E-31), DNMT3A+EZH2 (P=1.5E-13), DNMT3A2+EZH2 (P=1.1E-11), DNMT3B1+EZH2 (P=1.9E-185), DNMT3B2+EZH2 (P=9.4E-107), DNMT3B3+EZH2 (P=2.3E-68). Again, DNMT3B1 and DNMT3B2, which are alternative splicing isoforms differing by the presence of one exon, both in the presence and absence of EZH2 co-transfection, showed the lowest P-values, all less than 1E-100. From these data, we conclude that our list of 5,912 CpGs is indeed enriched for CpGs with substantially increased methylation when DNMTs or EZH2 were overexpressed, with DNMT3B1 and DNMT3B2 appearing to have the strongest impact on the DNA methylation levels at these sites.

Based on these data, we further investigated the altered DNA methylation in the DNMT3B1 and DNMT3B2 overexpression experiments. Because these splice isoforms differ by only one exon coding for 21 amino acids in a linker region, we suspected that they would share many targets. To identify the CpGs targeted by DNMT3B1 and DNMT3B2 in prostate tumors, we examined the list of CpGs that were hypermethylated in prostate tumors and in the overexpression experiments. Specifically, we looked for overlaps in the list of CpGs with 5% or greater increase in methylation compared to the mock in the DNMT3B1 (1267 CpGs), DNMT3B1+EZH2 (1322 CpGs), DNMT3B2 (1261 CpGs), and DNMT3B2+EZH2 (1235 CpGs) overexpression experiments. Four hundred and thirty eight CpGs were represented in all 4 lists and an additional 425 CpGs were represented in 3 of the 4 lists. We performed two permutation tests to determine the likelihood of our results. In the first permutation test, we generated 4 lists of CpGs (1267, 1322, 1261 and 1235 CpGs, respectively) drawn randomly from the whole list of 26,333 CpGs and counted the number of incidences where there was an overlap of 438 CpGs in all 4 lists. It was never observed in the 10,000 iterations. In our second permutation test, we repeated the first permutation test but changed the criteria to observing at least 863 CpGs overlapping in 3 of the 4 lists. This too was never observed in 10,000 iterations. This provided further evidence that the differentially methylated CpGs in the DNMT3B1 and DNMT3B2 overexpression experiments indeed significantly deviated from random sampling, and are likely to be those that are specifically, directly or indirectly, targeted by these methyltransferases.

Alterations in DNA methylation have been shown to play a role in tumorigenesis and cancer progression in many malignancies. Until recently, technical limitations have restricted these findings to either characterization of a handful of candidate loci or of overall abundance of 5-methylcytosine in the genome. No prior study has examined the methylation profiles of normal prostate tissue necessary to determine the methylation changes that occur during or as a result of tumorigenesis. Here, we present quantitative DNA methylation levels at more than 26,000 loci across 14,000 gene promoters. Because we assayed 95 cancers and 86 benign adjacent prostate tissues in parallel at CpGs specifically enriched at gene promoters, we were able to show that 43% of gene promoters represented in our assay had a tumor-specific methylation change. In addition to confirming methylation changes seen in previously published candidate loci studies, we also identified thousands of novel changes, including a set of hypermethylated loci more strongly predictive of prostate cancer than GSTP1. Our data show that DNA methylation changes in prostate cancer occur on a broad scale, at many loci throughout the genome.

DNA methylation alteration has been observed in early cancers and precursor lesions suggesting that methylation changes drive malignant initiation rather than tumor progression. Our observations are largely consistent with this hypothesis. If the acquisition of DNA methylation alterations continues throughout tumor progression, variation in methylation profiles should be observed in tumors of different histological grades and clinical outcomes. Although we detected more heterogeneity among tumors than among benign adjacent tissues, the vast majority of tumors fell in a single cluster and we did not observe obvious subclassifications, though some tumor samples did cluster with benign adjacent samples. We compared clinical outcomes of the donors of the tumors that clustered with benign adjacent tissues against the donors of the other tumors but did not observe any differences in Gleason grades or time-to-recurrence. However, from the little inter-tumor heterogeneity that did exist, we identified several dozen DNA methylation changes that correlated with patients' time-to-recurrence.

The fact that we observed changes at a very specific subset of CpG sites across most tumors, rather than a global DNA methylation deregulation or instability, suggests a common mechanism among prostate cancers. This specificity in target sites was particularly apparent in gene promoters assayed by multiple probes and by the PyroMark assay. The case of GSTP1 illustrates this point well, where the methylation changes were highly context dependent: only the CpG island overlapping the transcriptional start site was hypermethylated. Based on these findings, we suspect that cellular processes involved with targeted CpG methylation regulation are themselves misregulated or altered in early tumor initiation. The most likely candidates are DNMTs and DNMT-interacting proteins. In support of this hypothesis, we observed significant correlations between the gene expression levels and levels of global hypermethylation for several of these candidates. In vitro experiments in normal prostatic epithelial cells confirmed that overexpression of DNMT3B1 and DNMT3B2 leads to the hypermethylation of a subset of the prostate tumor-specific changes. These data, together with previous observations, strongly suggests that dysregulation of DNMTs and possibly DNMT-interacting proteins are among the earliest events in tumorigenesis.

While we did not address the mechanism for the observed decreased methylation of some CpGs in tumors, there are three likely possibilities. First, there may be aberrations in the maintenance DNA methyltransferase gene, DNMT1. Although we did not observe a decrease in the DNMT1 transcript level, there may be translational dysregulation of this gene or mutations that leads to decreased activity. Decrease in DNMT1 activity may lead to improper maintenance and gradual loss of methylation with every DNA replication. However, this would likely lead to a global loss rather than targeted loss at particular CpGs, and therefore, is the least likely scenario. A second possibility is the dysregulation of a direct or indirect DNA demethylase. Finally, the targeted hypomethylation may be the result of dysregulation of an interacting protein of DNMT1 or the hypothetical DNA demethylase.

By approaching DNA methylation in cancer from a genomic perspective, we were able to gain new insights into the underlying biology of prostate cancer, as well as discover novel markers for more accurate diagnosis of the disease. In addition, this is the first study comparing methylation in prostate cancer to benign adjacent tissue. Expanding an integrative analysis to include DNA methylation data along with gene expression and CNV data provides a better understanding of prostate cancer biology, and biomarkers for use in a clinical setting.

### Materials and Methods

Sample collection and preparation. All prostate samples used for this study were collected at the Stanford University Medical Center between 1999 and 2007 with patient's informed consent under an IRB-approved protocol. Multiple tissue samples were harvested from each prostate, flash frozen and stored at −80° C. Sections of each prostate tissue sample were evaluated by a genitourinary pathologist. The tumor and non-tumor areas were marked and contaminating tissues were trimmed away from the block as described previously. Tumor samples in which at least 90% of the epithelial cells were cancerous, and non-tumor samples having no observable tumor epithelium, were selected for extraction of DNA and RNA.

Primary prostate cell culture and transfection assays. A primary culture of human prostatic epithelial cells (E-PZ-231) was established from benign tissue of the peripheral zone of the prostate of a 56 year-old man who underwent radical prostatectomy to treat prostate cancer. Using previously described methods, primary cultures were serially passaged. When tertiary passage cells were about 50% confluent, they were fed Complete PFMR-4A medium (Peehl 2002) without gentamycin until they reached ˜85% confluency. Cells in each 60-mm, collagen-coated dish were then transfected with 10 μg of plasmid DNA using Lipofectamine 2000 (Invitrogen) according to the manufacturer's instructions. After 48 hours, cells from three 60-mm dishes per condition were dissociated with TrypLE Express (Invitrogen), centrifuged, and snap-frozen in liquid nitrogen. These cell pellets were then used for DNA isolation.

Nucleic acid isolation. DNA and RNA were isolated from tissue samples or cell cultures using Qiagen AllPrep DNA/RNA mini kit (Qiagen) following the manufacturer's protocol, with the exception of the RNA from primary prostate cell cultures. This RNA was isolated with Trizol Reagent (Invitrogen) according to the manufacturer's instructions.

Sodium bisulfite conversion. Sodium bisulfite conversion of genomic DNA was performed using the EZ-96 DNA Methylation Kit (Deep-Well format) (ZymoResearch). The conversion was completed using the alternative incubation protocol for Illumina Infinium Methylation Assay, as described by the manufacturer.

Methylation analysis by Illumina Infinium HumanMethylation27. Five hundred ng of sodium bisulfite-converted genomic DNA from patient samples or cultured cells were assayed by Infinium HumanMethylaton27, RevB Beadchip Kits (Illumina). The assay was performed using the protocol as described by the manufacturer.

Beta score calculations, quality filtering and batch normalization. HumanMethylation27 array results were initially extracted and analyzed using Illumina BeadStudio software with the Methylation Module v3.2. Beta scores were calculated manually using values exported from BeadStudio. For each probe intensity value, we subtracted the median negative background control probe value based on the color channel. The beta score was calculated using the background subtracted intensity values as: β=Intensity_{Methylated}/(Intensity_{methylated}+Intensity_{Unmethylated}). Any negative beta scores were converted to a zero. Any beta scores with an associated detection p-value of greater than 0.01 were converted to “missing values”. To correct for any array-by-array variation, we imputed all missing values using KNN Impute, then performed normalization using the ComBat R-package (Johnson et al. 2006). All previously imputed values were converted back to “missing values” for subsequent analyses.

To remove CpG probes with potentially problematic hybridization, we performed BLAT on all 27,578 probe sequences against the GRCh27/hg19 build of the human genome. One thousand and twenty eight probes showed questionable mapping and therefore were removed from analysis. We also identified 217 probes that included a SNP of greater than 3% minor allele frequency within 15 bp of the assayed CpG. These probes were also rejected with consideration to potential variation in probe hybridization due to the common SNP.

Clustering. Prior to each hierarchical clustering, the beta scores were mean centered. Hierarchical clustering of the arrays was done using the software Cluster 3.0 with Average Linkage. Because these datasets were too large to cluster the genes by Cluster 3.0, gene clustering was done using XCluster, available through the Stanford Microarray Database, using non-centered Pearson Correlation to perform the hierarchical clustering.

Significance Analysis of Microarray (SAM). Each SAM was performed as described in the software manual. The data were analyzed using the latest version of SAM available at the time of this manuscript preparation, which was version 3.09c. SAM was implemented using R version 2.10.0.

Prediction Analysis of Microarray (PAM). Prior to PAM, the CpGs were sorted by standard deviation across all tumors and benign adjacent tissue. To improve statistical power, only CpGs which had a standard deviation of 0.04 or greater were analyzed. PAM was performed as described in the software manual. The data were analyzed using the latest version of PAM available at the time of this manuscript preparation, which was version 2.11. PAM was implemented using R version 2.10.0. Based on visual examination of the training errors and the cross-validation results, we set the shrinkage threshold to 10.5.

PyroMark assays. PyroMark assays were performed at the Stanford Protein and Nucleic Acid Facility using the manufacturer's recommended protocol (Qiagen). For each target region, 3 primers were used: a forward and reverse PCR primer and a sequencing primer.

TaqMan gene expression assay. Expression levels of genes encoding several DNMT and DNMT-interacting proteins, as well as beta-2-microglobulin as an endogenous control, were measured in 10 benign adjacent and 36 tumor samples by TaqMan Gene Expression Assay. We used the following Applied Biosystems inventoried assays with FAM/MGD labeled probes (Assay ID in parentheses): DNMT1 (Hs00945900 g1), DNMT3A (Hs00173377 ml), DNMT3A2 (Hs00601097 ml), DNMT3B (Hs01003405 ml), DNMT3L (Hs01081364 ml), EZH2 (Hs01016789 ml) and the Human B2M (beta-2-microglobulin) Endogenous Control. Twenty five ng of cDNA were assayed in triplicate for each target, using the protocol as described by the manufacturer, on the ABI PRISM 7900HT instrument.

The results were analyzed using the ABI SDS 2.4 and ABI RQ Manager 1.2.1 software. Briefly, the average CT and delta-CT were calculated for each DNMT and EZH2. By integrating the average CT value from the B2M CT, we calculated the delta-delta-CT. All sample delta-delta-CT values were normalized to that of a tumor sample PC625T to generate an RQ value. To present the RQ value as a positive value, we added 5 to each RQ value.

Expression vectors. The pcDNA3/Myc-EZH2 construct was a generous gift from A. Chinnaiyan (Okano et al. 1999). The pcDNA3/Myc-DNMT3A, pcDNA3/Myc-DNMT3A2, pcDNA3/Myc-DNMT3B1, pcDNA3/Myc-DNMT3B2 and pcDNA3/Myc-DNMT3B3 constructs were a generous gift from A. Riggs (Chen et al. 2005).

What is claimed is:

1. A method for diagnosis of prostate cancer, the method comprising:
determining the presence of a change in methylation state in one or more biomarker(s) set forth in Table 1 in a sample suspected of comprising prostate cancer cells,
wherein the presence of altered methylation relative to a control sample is indicative of the presence of prostate cancer cells in the sample.

2. The method of claim 1, genomic DNA is isolated from sample suspected of comprising prostate cancer cells.

3. The method of claim 2, wherein said sample is a biopsy sample.

4. the method of claim 2, wherein said sample is a blood sample.

5. The method of claim 2, wherein said sample is a urine sample.

6. The method of claim 2, wherein said sample is seminal fluid sample or a component of seminal fluid.

7. The method of claim 1, wherein at least 5 biomarkers are screened.

8. The method of claim 1, wherein said screening comprises the step of converting unmethylated cytosine resides in said genomic DNA to uracil in a converted DNA sample.

9. The method of claim 6, wherein said converted DNA sample is amplified.

10. The method of claim 7, wherein said amplified DNA is sequenced to determine the methylation status of said one or more biomarkers.

11. The method of claim 7, wherein said amplified DNA is hybridized to a probe or an array of probes to determine the methylation status of said one or more biomarkers.

12. The method of claim 9, wherein the probe is attached to a solid surface.

13. The method of claim 9, wherein said probe is attached to a bead.


SEQUENCE LISTING (155 sequences)

SEQ ID NO: 1 - DNA, 50 bp, H. sapiens
gggtgcaggtgcacgctggcacttgagacgaatcttgaggaggcgaatcg

SEQ ID NO: 2 - DNA, 50 bp, H. sapiens
cgcccagggcgggtgtccccaccctcagcgagctcctctgcgacttctca

SEQ ID NO: 3 - DNA, 50 bp, H. sapiens
ggtggtgggagacgcagagtgcggcaagacggcgctgctgcaggtgttcg

SEQ ID NO: 4 - DNA, 50 bp, H. sapiens
cgccccggggcgcgcagttggaggcacatccccaccgcactctccaccct

SEQ ID NO: 5 - DNA, 50 bp, H. sapiens
aggtagtttctggagcccgatggcaggggcccattcagtgcgtttctgcg

SEQ ID NO: 6 - DNA, 50 bp, H. sapiens
cgccagcgcctgttcgttggcccacatggcccactcgatctgccccatgg

SEQ ID NO: 7 - DNA, 50 bp, H. sapiens
tgacagttgttggccccaaagttaagcgcgatttgtacggcctttacacg

SEQ ID NO: 8 - DNA, 50 bp, H. sapiens
cgccttctggaaagtttagaaagtgagccacgaaagagaggccacatttc

SEQ ID NO: 9 - DNA, 50 bp, H. sapiens
gatttgctcaggaagccgaccttcacaccttgtcctgctattaatagacg

SEQ ID NO: 10 - DNA, 50 bp, H. sapiens
gcatatctaagaggctgaacatgaatccacagatcaggtacctctgcacg

SEQ ID NO: 11 - DNA, 50 bp, H. sapiens
actggtgttccttctgaagctgacatctggcctcagctgggactctggcg

SEQ ID NO: 12 - DNA, 50 bp, H. sapiens
ggctacattggctggcaggggctgagcagcggtgagcctggctggcttcg

SEQ ID NO: 13 - DNA, 50 bp, H. sapiens
cgccagcgcccctacggattagcccccagggatctctgagcctggtatcc

SEQ ID NO: 14 - DNA, 50 bp, H. sapiens
tgcggcagccaataggagccgctctcctgaacattcagaggatgggtgcg

SEQ ID NO: 15 - DNA, 50 bp, H. sapiens
cggcactgtgactccaggaacactcacatccagccccttggggcaggagg

SEQ ID NO: 16 - DNA, 50 bp, H. sapiens
gaggagttccagtcaccgagcgaggggcgcaagggtgggtgcatcctgcg

SEQ ID NO: 17 - DNA, 50 bp, H. sapiens
tttcagagcatagctttctcaactatggcccggacgaagcagacagctcg

SEQ ID NO: 18 - DNA, 50 bp, H. sapiens
cgggtcatcaccccaggccccggggcagcccagaaccaggacaggaagac

SEQ ID NO: 19 - DNA, 50 bp, H. sapiens
acaggaccgagtccttggctgcctgtggagctcctgtgccagcagctgcg

SEQ ID NO: 20 - DNA, 50 bp, H. sapiens
atcttttggggccctcggcttgggttgggcccctgccagttgggcgagcg

SEQ ID NO: 21 - DNA, 50 bp, H. sapiens
ttttaaagacccgacaaactgggaaattgaccgagttctgtttctccccg

SEQ ID NO: 22 - DNA, 50 bp, H. sapiens
cgctttcttggaggacagccccagagccatggtggtctggacaaagctcg

SEQ ID NO: 23 - DNA, 50 bp, H. sapiens
cggacctgccagccccagggaacaaaagcggagcccgctcgccctctact

SEQ ID NO: 24 - DNA, 50 bp, H. sapiens
tgtgtatttgagacagggaactgttcctgtccccagccgatgaccagacg

SEQ ID NO: 25 - DNA, 50 bp, H. sapiens
cgcagacctatgatgtggagaactggatcgccaaaatagagacctcttgg

SEQ ID NO: 26 - DNA, 50 bp, H. sapiens
ttccacccttgcagggagcctgacactgagggctggcggcttttctggcg

SEQ ID NO: 27 - DNA, 50 bp, H. sapiens
cgggcgtccttctagaagcccatctcgctcacctgtgtggtcacccttgt

SEQ ID NO: 28 - DNA, 50 bp, H. sapiens
gctctagccgtcgaggagctgcctggggacggtacgtggcttaggggtcg

SEQ ID NO: 29 - DNA, 50 bp, H. sapiens
aggaccatcagcaacttcatggtgaggctgctgctgtcggtgttcagtcg

SEQ ID NO: 30 - DNA, 50 bp, H. sapiens
cgctcagccctggacggacaggcagtccaacggaacagaaacatccctca

SEQ ID NO: 31 - DNA, 50 bp, H. sapiens
cgggaccaaattaggggctgggagtttccagattgaaatgcgccctccac

SEQ ID NO: 32 - DNA, 50 bp, H. sapiens
gtctcaagggccagtgtcgggacagttgtcagcagggctccaacatgacg

SEQ ID NO: 33 - DNA, 50 bp, H. sapiens
cggccgggcccccaaaccttgcagtctcactcgccggtgagataatctgg

SEQ ID NO: 34 - DNA, 50 bp, H. sapiens
cgacatgccccggcaaccaagtcctggcctgggagcccaccctcagcccc

SEQ ID NO: 35 - DNA, 50 bp, H. sapiens
aaaaggcatctttgaactgcagctggggcatcatcctcaggcgtctgccg

SEQ ID NO: 36 - DNA, 50 bp, H. sapiens
cgagaggaagcaggtgttctcgataaaagcagcagccctaattttatggt

SEQ ID NO: 37 - DNA, 50 bp, H. sapiens
ctcgggggcctttggctccaggcaacttggggcaagcgtctcagttctcg

SEQ ID NO: 38 - DNA, 50 bp, H. sapiens
cgagcatggaactgagaaagtcctgtatagaggttaactatagagttgcc

SEQ ID NO: 39 - DNA, 50 bp, H. sapiens
cgcccctgtcctgggagtcccttggcccagacacccacctgacttagtgg

SEQ ID NO: 40 - DNA, 50 bp, H. sapiens
cgcctgcccgggaacatcctcctggactcaatcatggcttgtgtgagtgt

SEQ ID NO: 41 - DNA, 50 bp, H. sapiens
cgccgatcgccgacccacatccctgcgcccgcagccaggaccccctactt

SEQ ID NO: 42 - DNA, 50 bp, H. sapiens
ggattactgggtcacggtttcccaaggacatggaaacccttgctgaagcg

SEQ ID NO: 43 - DNA, 50 bp, H. sapiens
tgggggcccaggggtggcggctgcggcagggggtcccggggtcgggaccg

SEQ ID NO: 44 - DNA, 50 bp, H. sapiens
cgcccgcccaagcccagacctcggacctggttccaagcctgttcccgctg

SEQ ID NO: 45 - DNA, 50 bp, H. sapiens
cgggtcctttgactggcgtccagctgaccccaaccccggaccttcaaagt

SEQ ID NO: 46 - DNA, 50 bp, H. sapiens
ctttctccgtcggggtggatgggttggactttaggctccagcaagccccg

SEQ ID NO: 47 - DNA, 50 bp, H. sapiens
cggcgcctactgcgtaccaagcaccctctaagaaggacgaacacagctcc

SEQ ID NO: 48 - DNA, 50 bp, H. sapiens
tttctcacatgatttttcaggcactttcgcttttccatatataggagtcg

SEQ ID NO: 49 - DNA, 50 bp, H. sapiens
cgcacccccaggcactcaccccctgcccgagctgccgcctgagtaggtat

SEQ ID NO: 50 - DNA, 50 bp, H. sapiens
cgccagccgccagctgctgagtcacttttgtcaaagagtggcctcggccc

SEQ ID NO: 51 - DNA, 50 bp, H. sapiens
cgccccggccccgcgctgatgaaattgaggagctcacccagcacccttcc

SEQ ID NO: 52 - DNA, 50 bp, H. sapiens
cggggttctagaaatccgaggttctaagcctaggtgctccaataaaccca

SEQ ID NO: 53 - DNA, 50 bp, H. sapiens
cgggggcaataattctctaagagaactggagcccgaaagaggaatgaaaa

SEQ ID NO: 54 - DNA, 50 bp, H. sapiens
cgccctgggctcggtaacccccagccagcgtcccccagcccagctagcgc

SEQ ID NO: 55 - DNA, 50 bp, H. sapiens
cgacagcaggaccagctgtcctcacagcctcagatggctgagtctgagga

SEQ ID NO: 56 - DNA, 50 bp, H. sapiens
cggccccgctcgattcctggaatcttatttttggacctgctgccgcaagc

SEQ ID NO: 57 - DNA, 50 bp, H. sapiens
cggcctccgagccttataagggtggtcccgccccgctccgccccagtgct

SEQ ID NO: 58 - DNA, 50 bp, H. sapiens
tgtctgtgggctgggcagtgggctggatgacaccggctttgcaggcaccg

SEQ ID NO: 59 - DNA, 50 bp, H. sapiens
gtatggggtgatcttgggcttgtaaccgaatccaccagccgggcaggacg

SEQ ID NO: 60 - DNA, 50 bp, H. sapiens
cgcccaggagggccaccagatctgggagcttttcaactcaagcctcttca

SEQ ID NO: 61 - DNA, 50 bp, H. sapiens
cgggaggagaataaaactaaatgaccgtcaaaagtcaaggcttctgttcc

SEQ ID NO: 62 - DNA, 50 bp, H. sapiens
gatggctggctctagggaaggcatcagggcccctcagagttacctggacg

SEQ ID NO: 63 - DNA, 50 bp, H. sapiens
cctttacgcctttttatttgcggcggcttagcttggaaaacggtgttccg

SEQ ID NO: 64 - DNA, 50 bp, H. sapiens
acgggaagatgccgcgaggggcgtcattagggtaattgtgcccattaccg

SEQ ID NO: 65 - DNA, 50 bp, H. sapiens
cgggaaccacagagaaggaaaaagaagaaccacaagcgttttgagaaaca

SEQ ID NO: 66 - DNA, 50 bp, H. sapiens
ctaattgctcaacgtgggtgtagcacggattaggccttttacagcaagcg

SEQ ID NO: 67 - DNA, 50 bp, H. sapiens
tggactttgatcgccgagggctctctgctcttcagagtctgcttggaacg

SEQ ID NO: 68 - DNA, 50 bp, H. sapiens
cgccttcgtggccccaactcggcgctctgctatctctgatccggtgaaca

SEQ ID NO: 69 - DNA, 50 bp, H. sapiens
cggggggaaattccctaagaccgctgcgatcccggagcttgcacacccgc

SEQ ID NO: 70 - DNA, 50 bp, H. sapiens
ggttggggcgggggtgcagacacatcacggggcggtttggtatccatccg

SEQ ID NO: 71 - DNA, 50 bp, H. sapiens
cgcccactgcctgcacaagcctcaggcctatgggggtcactggccttggg

SEQ ID NO: 72 - DNA, 50 bp, H. sapiens
cgacccaagcagaccccactgtgttccaggagctgttccttgagagggat

SEQ ID NO: 73 - DNA, 50 bp, H. sapiens
gttgaaagactggtcgaaattacgcgggcatgagtcagcgcatccctacg

SEQ ID NO: 74 - DNA, 50 bp, H. sapiens
ctgcttggactccgcgtcagtccaggtggccttcaaggagactttgttcg

SEQ ID NO: 75 - DNA, 50 bp, H. sapiens
cgccgagcccggagttcaccactctattgcgggtgttcatggttcacagc

SEQ ID NO: 76 - DNA, 50 bp, H. sapiens
cggagaactgtggcatcccaggcccaccgtcttcaccagtagcagcccgc

SEQ ID NO: 77 - DNA, 50 bp, H. sapiens
gggagattcgggctggaacagcggtaatgggcacaattaccctaatgacg

SEQ ID NO: 78 - DNA, 50 bp, H. sapiens
ttacaactcttcattctgaagtgcgtgtagtgcccttgtctccagagacg

SEQ ID NO: 79 - DNA, 50 bp, H. sapiens
agggaggctgcgaacaacgggctgtttcagctccgagattttgcgatccg

SEQ ID NO: 80 - DNA, 50 bp, H. sapiens
cgacaaccagcaaatccccagagacaggtccctgggaattagctgcgccg

SEQ ID NO: 81 - DNA, 50 bp, H. sapiens
tcaaacgaacggagcaaaccctgggatcgtttcaaaggatttttaacccg

SEQ ID NO: 82 - DNA, 50 bp, H. sapiens
cctcgcccttcagagtaactcctgtggactctgagactctgggatattcg

SEQ ID NO: 83 - DNA, 50 bp, H. sapiens
cgcacagctttgtttaaaagtcccaggttgtgtggaggggcagcccaaag

SEQ ID NO: 84 - DNA, 50 bp, H. sapiens
aatatcccagagtctcagagtccacaggagttactctgaagggcgaggcg

SEQ ID NO: 85 - DNA, 50 bp, H. sapiens
cggactccagaccgccagctgagacctttagctcaactagtggttggcac

SEQ ID NO: 86 - DNA, 50 bp, H. sapiens
cctaccggcattgaaatacttatggataaagttctcgcaatggcttcacg

SEQ ID NO: 87 - DNA, 50 bp, H. sapiens
cggcctcagttcctaaaggtgaccagggaaaaactcaaggagcttctatc

SEQ ID NO: 88 - DNA, 50 bp, H. sapiens
cggacaggcaccacgctaatctggcatctcccaggcccattaccggatcg

SEQ ID NO: 89 - DNA, 50 bp, H. sapiens
gtggcagaggccagagcccagaggcgcagcccgggcagctaggagggtcg

SEQ ID NO: 90 - DNA, 50 bp, H. sapiens
ggaactgttcgggttcctgcaggacgtcacagatggtgttcaccatctcg

SEQ ID NO: 91 - DNA, 50 bp, H. sapiens
cgggcctcacacaggccgactctgggtcgtcagttcctcatcagctcgaa

SEQ ID NO: 92 - DNA, 50 bp, H. sapiens
cgcccaaccaccgagtggtggagccaggactcaactcaagtctgccccac

SEQ ID NO: 93 - DNA, 50 bp, H. sapiens
ggtcaggttgagaccccagcccagcaagatgggcacggaaatgttgggcg

SEQ ID NO: 94 - DNA, 50 bp, H. sapiens
ttgcgggctgactgaccagtgtgctaatcacatctgcatttggggcctcg

SEQ ID NO: 95 - DNA, 50 bp, H. sapiens
ccaggctctgccagatctcaaagtgagaaccttgagggatgactgaaccg

SEQ ID NO: 96 - DNA, 50 bp, H. sapiens
cggaacccgaggggggtcttaactagtcatagtctcaggaccacacatct

SEQ ID NO: 97 - DNA, 50 bp, H. sapiens
gcctgcgaaggcatctcagtatgtgtaatgcatcccctctttttttcccg

SEQ ID NO: 98 - DNA, 50 bp, H. sapiens
cgggaagatacagcatgagtttctgtccaagaggttttagctgtaatgaa

SEQ ID NO: 99 - DNA, 50 bp, H. sapiens
cgccgccgcccatccgacctgccccacaggtcctggccacccagccaccg

SEQ ID NO: 100 - DNA, 50 bp, H. sapiens
cgacattggggtggggaaccctgacattcactgattagtcaagactgggt

SEQ ID NO: 101 - DNA, 50 bp, H. sapiens
cgcaggtggggataagagtgagtgagtcaataaagaagaaaattgcccca

SEQ ID NO: 102 - DNA, 50 bp, H. sapiens
taatgacctactgggtttcaggcatgatatgctcattacctctttaatcg

SEQ ID NO: 103 - DNA, 50 bp, H. sapiens
aatgacaatggctgctgagaattcctccttcgtgacacagtttatcctcg

SEQ ID NO: 104 - DNA, 50 bp, H. sapiens
cgggatgtagttcaaccctagaagccagatctggtgtctggaaagcaggt

SEQ ID NO: 105 - DNA, 50 bp, H. sapiens
cgccttcaccagatacctccaggggcaagagtccactgaggttacagcgc

SEQ ID NO: 106 - DNA, 50 bp, H. sapiens
cgggggcatgcaaaccacagttgacctactagctgaagcagtgataaaag

SEQ ID NO: 107 - DNA, 50 bp, H. sapiens
cgcagacactatgctgcctcccatggccctgcccagtgtgtcctggatgc

SEQ ID NO: 108 - DNA, 50 bp, H. sapiens
cggcccacccagaaagtgaaatcaaaacaggaagtcaccaggggtgactg

SEQ ID NO: 109 - DNA, 50 bp, H. sapiens
cgtgggatggatcaaaagggacagagaactctttttgaaagttgtaataa

SEQ ID NO: 110 - DNA, 50 bp, H. sapiens
ccctatgtggaaaatgcataatctctaacataatgacggggtcaacctcg

SEQ ID NO: 111 - DNA, 50 bp, H. sapiens
cggagcccctgtagtttgccctcttcatttattttcagtggatttccacg

SEQ ID NO: 112 - DNA, 50 bp, H. sapiens
cgtgcagaacctgtgtttacagccatgataatgcatcttgggggttcctg

SEQ ID NO: 113 - DNA, 50 bp, H. sapiens
ttgaatgtgtgctttccacagtttctgatcctcagctcccactctcttcg

SEQ ID NO: 114 - DNA, 50 bp, H. sapiens
cagaagaggccacatctgcttcctgtaggccctctgggcagaagcatgcg

SEQ ID NO: 115 - DNA, 50 bp, H. sapiens
cgctgcagttgagaactagcagatcctattggtagtgccctgtggcccac

SEQ ID NO: 116 - DNA, 50 bp, H. sapiens
cgtgttattgtgaatgccacacccataccagcagctgggctgggagatgc

SEQ ID NO: 117 - DNA, 50 bp, H. sapiens
cgatctttgctgagtgtctatctagcctcagatttataagtctgggtgtg

SEQ ID NO: 118 - DNA, 50 bp, H. sapiens
cgtgccagacagcttaccagggtcagtcacgagcccagagtcaaaccctg

SEQ ID NO: 119 - DNA, 50 bp, H. sapiens
gtcacgtggaatcatctaagtggtgagcagcatttctgccccctttatcg

SEQ ID NO: 120 - DNA, 50 bp, H. sapiens
cctgttttcgcctcaatgttgcattttctgagaccactctagctgtcacg

SEQ ID NO: 121 - DNA, 50 bp, H. sapiens
ctttacagtgtatcctaaatctgatcacttctcatcatctctaggccacg

SEQ ID NO: 122 - DNA, 50 bp, H. sapiens
agaccttgaccattttgaggctgtccttctgcacaaatatggaaattccg

SEQ ID NO: 123 - DNA, 50 bp, H. sapiens
cggaccagcactccactgtgggtccaaggatgagctccaaagagcccagt

SEQ ID NO: 124 - DNA, 50 bp, H. sapiens
cggtgatggttcaggttgtgtttctgggttcattctggaagctcccccaa

SEQ ID NO: 125 - DNA, 50 bp, H. sapiens
gtctccggtgtggcaggcaggtttttccaggcagctggcaggtgtgctcg

SEQ ID NO: 126 - DNA, 50 bp, H. sapiens
agtgcttggggatgcaggtccttgcgataaggggccgataccacctcccg

SEQ ID NO: 127 - DNA, 50 bp, H. sapiens
tttgctccttaatcactgtcacagacaattgatactgccattgatactcg

SEQ ID NO: 128 - DNA, 50 bp, H. sapiens
cggggagacacacagataagtagaccattcaaaagtaggtttatgctaga

SEQ ID NO: 129 - DNA, 50 bp, H. sapiens
actagcctctctctctactattaagctggcttaccatcttatgtcattcg

SEQ ID NO: 130 - DNA, 50 bp, H. sapiens
cgagttttatacttaatttgccaggggttcgctgcagaagcggcagagac

SEQ ID NO: 131 - DNA, 50 bp, H. sapiens
cacatagatattcatcatagaactgccatgatactcccatgtttggctcg

SEQ ID NO: 132 - DNA, 50 bp, H. sapiens
gtgtgtgcgggcccaggacttactcgaagggcgcacttcttgggaatgcg

SEQ ID NO: 133 - DNA, 50 bp, H. sapiens
cgggctagagtcatcctgactcggccacccctgcagctgggcaaacttgt

SEQ ID NO: 134 - DNA, 50 bp, H. sapiens
tacctgaagaggatcaaagacacaccctggctatggcaggtttctcctcg

SEQ ID NO: 135 - DNA, 50 bp, H. sapiens
cgaagctttgtgaagatcacagctaccttaatgggagagaaagctcattt

SEQ ID NO: 136 - DNA, 50 bp, H. sapiens
cgcccggcctatcgtgccctttcaacagatgaagaaactggtgagtttaa

SEQ ID NO: 137 - DNA, 50 bp, H. sapiens
cgagtgggattcatgacaacaatctgcaaaggaagaaactgaggctcagt

SEQ ID NO: 138 - DNA, 50 bp, H. sapiens
atcagagcctcctaaatctgttcatgtcacactgtcaggtttgggctacg

SEQ ID NO: 139 - DNA, 50 bp, H. sapiens
ctggtgacagctgtgaatctactagaacactacacatagccacaaaatcg

SEQ ID NO: 140 - DNA, 50 bp, H. sapiens
tactggtgcttccacctgccttggtctgagttgcagtccatggggcagcg

SEQ ID NO: 141 - DNA, 50 bp, H. sapiens
agacatgcagaatctcaggctttaatccagaacttctgattcagaatccg

SEQ ID NO: 142 - DNA, 50 bp, H. sapiens
ccatgaaggacttctcagatgtcatcctctgcatggaggcaacagaatcg

SEQ ID NO: 143 - DNA, 50 bp, H. sapiens
ccttggggctctgacaggtaggacccagcagggcgtggagccaggcaacg

SEQ ID NO: 144 - DNA, 50 bp, H. sapiens
cggcacccactctcgagacgtccgtccgcaccccagaactcgggcccaag

SEQ ID NO: 145 - DNA, 50 bp, H. sapiens
cggtggacaaaatgggaaaagctcagaaacttggtgttgaaatcggacct

SEQ ID NO: 146 - DNA, 50 bp, H. sapiens
cgcacctgcgcacaaaagaccacagtgtgagacacactcagggaaagcct

SEQ ID NO: 147 - DNA, 50 bp, H. sapiens
tttccctaggttggccgatttgatcaactcgtaggccttcttcaaggacg

SEQ ID NO: 148 - DNA, 50 bp, H. sapiens
agaaacctgcccaaaatggattaagtctcatctgtacattccccatgtcg

SEQ ID NO: 149 - DNA, 50 bp, H. sapiens
cgctgatagacatcaggtgacaggaaatcagtagcttctgctaccttggg

SEQ ID NO: 150 - DNA, 50 bp, H. sapiens
tgcagacgtccccacagagggcagtgccgaggacagtgtgtgtgcagacg

SEQ ID NO: 151 - DNA, 50 bp, H. sapiens
atggcacggtgctgcctcttgatgaccaggtggacagtgaggccatctcg

SEQ ID NO: 152 - DNA, 50 bp, H. sapiens
tgatctcaaccacacatggatgggacctctggttcaagcagaagaatgcg

SEQ ID NO: 153 - DNA, 50 bp, H. sapiens
cgttctgcactgattcattgtgtggtcttgagcaagttgtagagcttctc

SEQ ID NO: 154 - DNA, 50 bp, H. sapiens
cggaagcaactaagaaatgtcaagagtgccattttggaatcagagaagtc

SEQ ID NO: 155 - DNA, 50 bp, H. sapiens
cgcccccgtttcaacactaggcagaggccccagtcctgccacccgcaggc